(19) United States
(12) Patent Application Publication
(10) Pub. No.: US 2025/0121010 A1
(43) Pub. Date: Apr. 17, 2025

(54) A BIOMARKER INDICATING THE THERAPEUTIC EFFICACY OF EXTRACELLULAR VESICLE (EV)- PREPARATIONS

(71) Applicant: UNIVERSITÄT DUISBURG-ESSEN, Essen (DE)

(72) Inventors: Bernd GIEBEL, Köln (DE); Tobias TERTEL, Essen (DE); Simon STAUBACH, Essen (DE); Peter HORN, Essen (DE)

(21) Appl. No.: 18/684,552

(22) PCT Filed: Aug. 18, 2022
(86) PCT No.: PCT/EP2022/073109

§ 371 Date: Feb. 16, 2024

(30) Foreign Application Priority Data
Aug. 18, 2021 (DE) 10 2021 121 463.7

(51) Int. Cl. A61K 35/28; C12N 5/0775; G01N 33/68

(52) U.S. Cl. CPC A61K 35/28; C12N 5/0663; G01N 33/6872; G01N 2333/70596

(57) ABSTRACT

The present invention is based on a correlation found for the presence of the biomarker protein Cytotoxic T-lymphocyte protein 4, also known as CD152 or CTLA-₄, on or in EVs, to their immunomodulatory efficacy. Hence, the invention provides a method for assessing the therapeutic efficacy of an EV or an EV-preparation by detecting the biomarker CTLA-₄ in such EVs or EV-preparations. Furthermore, the invention provides a method for assessing the therapeutic efficacy of an EV or an EV-preparation by detecting at least one biomarker selected from the group of CTLA-₄, PD1, PD-L1 and PD-L2 in the EV or EV-preparation. The method of the invention allows for an evaluation of the immunomodulatory efficacy of said EV preparations without the need of performing laborious functional in vivo or in vitro assays including mixed lymphocyte reaction assays. In three additional aspects of the invention, alternative methods for producing therapeutically active EVs or EV-preparations are provided. Further provided are therapeutically active EVs or EV preparations produced by the methods of the invention and their use in medicine, for example in the manufacturing of immunomodulatory medicaments.

### CROSS-REFERENCE TO RELATED APPLICATIONS

This application is a national stage application of PCT/EP2022/073109 filed 18 Aug. 2022, which claims priority to German Patent Application No. 10 2021 121 463.7 filed 18 Aug. 2021, the entire disclosures of each application are herein incorporated by reference.

### SEQUENCE LISTING

The instant application contains a Sequence Listing which has been submitted electronically in XML format and is hereby incorporated by reference in its entirety. Said XML copy, created on 7 Feb. 2024, is named 0335-0014-PCT-US Sequence Listing.xml and is 26,535 bytes in size.

### FIELD OF THE INVENTION

The present invention is based on a correlation found for the presence of the biomarker protein Cytotoxic T-lymphocyte protein 4, also known as CD152 or CTLA-4, on or in EVs, to their immunomodulatory efficacy. Hence, the invention provides a method for assessing the therapeutic efficacy of an EV or an EV-preparation by detecting the biomarker CTLA-4 in such EVs or EV-preparations. Furthermore, the invention provides a method for assessing the therapeutic efficacy of an EV or an EV-preparation by detecting at least one biomarker selected from the group of CTLA-4, PD1, PD-L1 and PD-L2 in the EV or EV-preparation. The method of the invention allows for an evaluation of the immunomodulatory efficacy of said EV preparations without the need of performing laborious functional in vivo or in vitro assays including mixed lymphocyte reaction assays. In three additional aspects of the invention, alternative methods for producing therapeutically active EVs or EV-preparations are provided. Further provided are therapeutically active EVs or EV preparations produced by the methods of the invention and their use in medicine, for example in the manufacturing of immunomodulatory medicaments.

### DESCRIPTION

Extracellular Vesicles (EVs) are a group of lipid-bilayer enclosed nano- and microbodies that are released from all eukaryotic and prokaryotic cells. They can be found in various different biological fluids such as serum, plasma, urine, saliva, breast milk, amniotic fluid, ascites fluid, cerebrospinal fluid and bile. The main function of EVs lies in cell-cell signalling, a task that EVs mediate both by their surface components and by the delivery of cargo molecules such as proteins, nucleic acids, lipids and other metabolites.¹⁻³

In the last decade, the scientific community has shown an exponentially increasing interest in EV-research with roughly 2500 publications in the year 2018 alone. As a consequence, the knowledge in this field is rapidly evolving and, due to the influence of researchers from many different research areas, there is currently a non-standardization in terminology. The following sections discusses the most widespread definitions as they are also applied in this document.⁴

EVs are commonly categorized into three major populations: exosomes, microvesicles and apoptotic vesicles. These populations differ in their size, biogenesis and method of cellular release. Microvesicles are generated by plasma membrane budding and usually feature a diameter in the range of 100-1000 nm. Apoptotic vesicles, the larger ones also known as apoptotic bodies, originate by fragmentation of cells and have diameters starting in the same range than microvesicles up to several μm. Exosomes exhibit a diameter of 70-150 nm and are formed by inward budding of the late endosomal limiting membrane into the endosomes lumen to create intraluminal vesicles (ILVs). Endosomes containing ILVs are termed multivesicular body (MVB) or multivesicular endosomes. A proportion of the MVBs can fuse with the plasma membrane and release their ILVs as exosomes into the extracellular environment. Due to similarities in their surface structures, size and density, Exosomes, Microvesicles, and small Apototic Vesicles being smaller than 150 nm cannot be experimentally discriminated. For being precise most scientist agreed to decipher preparations with extracellular vesicles consequently in academia the collection of extracellular vesicles with sizes below 200 nm simply as small EVs (sEVs). Especially in the industry, the term “exosome” is used as a synonym for sEVs. In the following we stick to the academic nomenclature. EVs are assembled in a cell type specific manner and in addition to more generally occurring molecules including lipids and proteins contain cell type specific molecules that can contribute to their functions as intercellular communication vehicle.²⁻⁵

Recognized as intercellular communication vehicles that depending on their origin can mediate therapeutic functions, the interest in EV-research is raising exponentially. EV serve as a new class of biomarkers and are increasingly used for diagnostic purposes. As their composition is specific for the tissue or cells that they are originating from EVs can be used as biomarkers for different cancer types such as lung, breast, colorectal, and prostate cancer. Moreover, equipped with a tropism towards selected target cells they can serve as drug delivery vehicles.⁶ Furthermore, depending on their origin also native EVs provide therapeutic functions such as EVs harvested from supernatants (conditioned media) of mesenchymal stem/stromal cells (MSCs).⁷

MSCs are of particular interest in EV research. They can be derived from various tissues including bone marrow, adipose tissue, and umbilical cord tissue. While MSCs can directly be used in therapeutic applications via transplantation, they perform many of their functions in paracrine fashion by the secretion of MSC-EVs. The therapeutic effect of MSC-EVs is based on cargo-transfer as well as the triggering of signalling pathways via cell surface interactions. Therapeutically, this can be used for mitigation or elicitation of immune responses, regulation of tissue inflammation, inhibition of apoptosis, minimization of oxidative stress, stimulation of wound repair, and promotion of angiogenesis.⁸

Even without any loading of additional drug-molecules, EVs feature innate immunomodulatory properties. Their effect in cell-cell communication is highly variable, depending on the cell type they are originating from. Early preclinical and clinical studies on EVs were performed on dendritic cell (DC) derived EV-populations. While the purpose of these studies was using EVs as anticancer agents, they demonstrated a great variation of immunomodulatory effects for different EV populations. Immature DC-EVs were shown to feature immunosuppressive, mature DC-EVs immunostimulatory properties. These contrary effects are also reflected in the surface markers expressed on the DC-EV populations, which differ in their concentration of MHC I and II, varying costimulatory molecules such as CD40, CD80, CD86 and CD54 or the surface expression of the immunoregulatory protein PD-L1.⁸

In preclinical studies, MSC-EV preparations have been used in diverse indications. These include the treatment of diseases and regenerative medicine, in which EVs are used for treating the lungs, kidney, liver, central nervous system, cartilage, bone, and heart. The function of the MSC-EVs depends amongst others on their respective MSC-EV origin cells. For example, both dental-pulp MSC-EVs and umbilical cord MSC-EVs may be used in vesicular repair, the former due to the inclusion angiogenesis-activating Jagged-1 ligand protein, the latter have been shown to carry platelet-derived growth factor-D that mediates tissue repair functions. Embryonic stem cell derived MSCs can feature a therapeutic effect in bone regeneration by expressing the membrane protein CD73 that is activating regenerative protein kinase B (AKT) and extracellular signal-related kinase (Erk) signalling.

Bone-Marrow (BM) derived MSCs are the most extensively researched EV-population. As shown in the case of retinal ganglion cells, these EVs can mediate regenerative effects by harboring the argonaute-2 protein. BM-MSC-EVs are particularly known for their immunomodulatory properties. Depending on their preparation method, BM-MSC-EVs can feature anti-inflammatory properties for example by expression of CCR2 receptors that quench the macrophage-recruiting CCL2 chemokine. Furthermore, TNF-α and IFN-γ stimulated BM-MSC have been demonstrated to produce EVs with prostaglandin E2 and cyclooxygenase 2 expression that exhibit anti-inflammatory properties.⁹

One way to evaluate the therapeutic properties of MSCs is based on determining the molecular composition of their cargo and surface structures. Such studies have shown that, besides preparation parameters such as tissue origin, cell culture conditions, isolation method and EV-population, the therapeutic efficacy of MSC-EVs is highly donor-dependent. Therefore, even “established” protocols do not necessarily yield reproducible EV properties. For example, identically prepared BM-MSC-EV preparations can feature a variation of almost two-orders of magnitude in the ratio of pro-inflammatory IFN-γ and anti-inflammatory IL-10.¹⁰

In order to use EV preparations in biomedical applications, they need to be produced with a consistent batch-to-batch quality. Current methods for generating EV preparations either do not consider the fluctuating properties of EV-preparations, or are hardly suitable for production-upscaling due to on time-consuming and resource-intensive steps. Therefore, there is an unmet need of quick methods to assess, whether EV exhibit the desired immunomodulatory profile. The current invention solves this problem by providing a simple and fast method for analysing the immunomodulatory efficacy of EV-preparations that is based on determining the presence of EV surface markers.

### BRIEF DESCRIPTION OF THE INVENTION

Generally, and by way of brief description, the main aspects of the present invention can be described as follows:
In a first aspect, the invention pertains to a method for assessing the therapeutic efficacy of an Extracellular Vesicle (EV) or an EV-preparation, comprising the following steps:
   (i) Providing the EV or EV-preparation;
   (ii) Detecting the presence or absence of an EV surface marker, preferably CTLA-4, associated with the EV or EV-preparation, optionally by using a binding-agent;
Wherein the presence of the EV surface marker associated with the EV or EV-preparation is indicative of a therapeutically sufficient efficacy of the EV or EV-preparation.
In a second aspect, the invention pertains to a method for producing an Extracellular Vesicle (EV) or EV-preparation with therapeutically sufficient efficacy, comprising the following steps:
   (i) Providing a secretory cell;
   (ii) Culturing the secretory cell in a culture medium (and under conditions) that allows for a secretion of EVs into the culture medium;
   (iii) Harvesting of the secreted EVs to obtain a harvested EV or EV-preparation;
   (iv) Performing the method of the first aspect of the invention with the harvested EV or EV-preparation to determine its therapeutic efficacy;
   (v) Selecting the harvested EV or EV-preparation with therapeutically sufficient efficacy.
In a third aspect, the invention pertains to a method for producing a therapeutically active Extracellular Vesicle (EV) or EV-preparation with therapeutically sufficient efficacy, comprising the following steps:
   (i) Providing a secretory cell;
   (ii) Enhancing in the secretory cell the secretion of therapeutically efficient EVs;
   (iii) Culturing the secretory cell in a culture medium (and under conditions) that allows for a secretion of EVs into the culture medium
   (iv) Harvesting of the secreted EVs to obtain a harvested EV or EV-preparation;
   (v) Performing the method of the first aspect of the invention with the harvested EV or EV-preparation to determine its therapeutic efficacy;
   (vi) Selecting the harvested EV or EV-preparation with therapeutically sufficient efficacy.
In a fourth aspect, the invention pertains to an Extracellular Vesicles (EV) preparation obtainable by the method of the second and third aspect of the invention.
In a fifth aspect, the invention pertains to a use of the Extracellular Vesicles (EV) preparation of the previous aspects of the invention, in the manufacturing of a medicament.
In a special sixth aspect, the invention pertains to a method for manufacturing an Extracellular Vesicle (EV) or EV-preparation with therapeutically sufficient efficacy, comprising the following steps:
   (i) Providing a secretory cell;
   (ii) Culturing the secretory cell in a culture medium that allows for the secretion of an EV into the culture medium;
   (iii) Sorting the secreted EV into at least two fractions, depending on the presence or absence of an EV surface marker that is associated with the EV;
Wherein a fraction of the at least two fractions is the Extracellular Vesicle (EV) or EV-preparation with therapeutically sufficient efficacy
In a special seventh aspect, the invention pertains to an Extracellular Vesicle (EV) preparation with therapeutically sufficient efficacy obtainable by the method of the first, second, third, and/or sixth aspect of the invention.
In a special eighth aspect, the invention pertains to a use of the Extracellular Vesicles (EV) preparation of any other aspects of the invention, in the manufacturing of a medicament.
In a special ninth aspect, the invention pertains to an EV or EV preparation according to any other aspect of the invention and/or a medicament according to any other aspect of the invention for use in the treatment of a condition or disease.
In a special tenth aspect, the invention pertains to a method for the treatment of a condition or disease, the method comprising administering to a subject in need thereof an EV, EV preparation and/or medicament according to any other aspect of the invention.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a method for assessing the therapeutic efficacy of an Extracellular Vesicle (EV) or an EV-preparation, comprising the following steps:
(i) Providing the EV or EV-preparation;
(ii) Detecting the presence or absence of an EV surface marker, preferably CTLA-4, associated with the EV or EV-preparation, optionally by using a binding-agent;
Wherein the presence of the EV surface marker associated with the EV or EV-preparation is indicative of a therapeutically sufficient efficacy of the EV or EV-preparation.

The term therapeutic efficacy in the context of the invention refers to either
(i) A pharmacological effect, that is triggered by administering the EV-preparation, that would not be present or that would be weaker when administering a comparable preparation that is not comprising EVs;
(ii) A ratio defined by the strength of the pharmacological effect that is triggered by administering the EV-preparation that is containing at least one EV, when compared to the strength of the pharmacological effect that would result by administering a comparable preparation that is not comprising EVs.

In case of the EV or EV preparations of the present invention, this pharmacological effect refers to immunomodulation. Immunomodulation comprises a stimulation, activation, suppression or inhibition, either complete or partially, of the immune response of an organism. Immune response in the context of the invention comprises a differentiation, activation or proliferation of cells associated with the innate or adaptive immune system, the release of compounds that have a regulatory function to the immune system, or the generation or release of antigen-recognizing molecules such as antibodies, T-cell receptors or fragments thereof.

The term “extracellular vesicle” (EV) in the context of the invention refers to a lipid vesicle that is secreted by cells into the extracellular space. In preferred embodiments of the invention, the extracellular vesicle (EV) is a lipid-based extracellular body. In more preferred embodiments of the invention, the EV is an apoptotic vesicle(s), microvesicle(s) and/or exosome(s). These subpopulations are differentiated by size, biogenesis, release pathways, composition, and function.

The size ranges that are used to categorize EVs refer to the hydrodynamic diameter of these lipid-based vesicles. The hydrodynamic diameter may be determined with dynamic light scattering or nanoparticle tracking analysis and is depending on the mobility of the droplets/vesicles in media. Hydrodynamic diameter in the context of the invention refers the diameter of a hypothetical sphere that features the same mobility as the measured particles. In case of dynamic light scattering measurements, it refers to the average intensity-based hydrodynamic diameter (Z-average). These values include a standard deviation of ±1σ. Preferably, hydrodynamic diameters, as referred to herein, are measured by Dynamic Light Scattering at 20° C. in aqueous solution at concentration of 1 mg/ml using a “Zetasizer Lab red label” of the company Malvern Panalytical (United Kingdom), preferably wherein the aqueous solution is a phosphate buffered saline solution (PBS).

In preferred embodiments of the invention, the size of the EVs is 20 to 7000 nm, preferably 30 to 6000 nm, most preferably 50 to 5000 nm. In this size range, EVs refer to apoptotic vesicles, also known as apoptotic bodies. The membrane of apoptotic vesicles of the present invention comprises phosphatidylserine and, optionally, membrane components from their parent apoptotic cells. The cargo of apoptotic bodies is selected from at least one component of the group consisting of cellular organelles, chromatin, both intact and fragmentations thereof, small amounts of glycosylated proteins and cytoplasmic constituents of their apoptotic parent cell.

In preferred embodiments of the invention, the size of the EVs is 20 to 1500 nm, preferably 30 to 1200 nm, most preferably 50 to 1000 nm. In this size range, EVs may be assigned to the subset of microvesicles. They feature a lipid-bilayer membrane that is dependent on their cellular origin. The components of this membrane are selected from at least one of the components of the group consisting of acyl carnitines, lysophosphatidylcholines, cholesterol esters, ceramides, sphingomyelins, lysophosphatidylglycerols, lysophosphatidylinositols and lysophosphatidylethanolamines. Microvesicles can further comprise proteins the form of cargo and/or in the microvesicle membrane depending on their parent-cell and the microvesicle function. In case of MSC-derived microvesicles, the membrane proteins are selected from at least one component of the group of tumor susceptibility gene 101 (TSG101), calnexin, CD44, CD63, CD71, CD73, the integrins ITGB3, ITGA2B, ITGA6, ITGA1, the Rab protein RAB27B, and/or membrane proteins that are associated with their parent cells such as the common MSC-surface proteins CD73, CD90 and CD105. The cargo-proteins of MSC-MVs are selected from the group consisting of nucleic acids, such as microRNA, and proteins, such as the angiogenic factors including IL-8, VEGF, TIMP-1, TIMp-2, growth factors including EGF and bFGF, cytokines including GRO, IGF-I, I-TAC, MCP-1, MMP-1, VEGF-D and cytoplasmic components of their secretory cells.

In preferred embodiment of invention, the size of the EVs is 20 to 200 nm, preferably 30 to 180 nm, most preferably 50 to 150 nm. In this embodiment, EVs refer to the subset of exosomes, also known as intraluminal vesicles (ILVs). Surface markers present on the exosomes of the present invention are selected from at least one component of a list consisting of Tsg101, CD9, CD63, CD81, CTLA-4 and flotilin. Proteins expressed/enriched on exosomes of the present invention, that originate from MSCs are selected from at least one component of a list consisting of the cell surface markers CTLA-4, CD81, CD63, CD9, the integrins ITGB3, ITGA2B and ITGA6, the Rab proteins RAB27B and RAB27A in addition to the aforementioned MSC-specific surface markers. Compared to their parent cells, the membrane of MSC-exosomes are enriched in lipids are selected from at least one component of a list consisting of fatty acids, cardiolipins, lysophosphatidylserines, lysophosphatidylglycerols and lysophosphatidylinositols.

In preferred embodiments of the invention, detecting the presence or absence of an EV surface maker in step ii) comprises detecting a (specific) binding between the binding-agent and the EV surface marker. The term “EV Surface marker” according to the invention refers to any functional and non-functional structure that is expressed on the intact surface of EVs, or is associated with EV-preparations. These structures include carbohydrates, proteins and lipids-based molecules and also comprise any of the aforementioned membrane components that are featured by apoptotic vesicles, microvesicles or exosomes.

In preferred embodiments of the invention, the EV surface marker associated with the EV or EV preparation is a compound or substance, preferably a protein, which is bound, or capable of binding, to the surface, preferably outer surface, of an EV. This shall include such compounds or substances which are bound to the surface, preferably outer surface, of an EV when the EV is secreted, but which are in context of the inventive method subsequently actively removed from the EV surface or completely separated from the EV in order to facilitate EV surface marker detection.

In another alternative or additional embodiment of the invention, which is also preferred, the term “EV surface marker associated with the EV or EV preparation” does not comprise any compound or substance, such as a protein, which is not bound, or not capable of binding, to the surface, preferably outer surface, of an EV. Such excluded compounds or substances may be soluble compounds or soluble substances.

In a preferred embodiment of the invention, the binding-agent is an antigen binding construct, such as a protein or nucleic acid, and preferably is a ligand molecule specific for the EV surface marker or derivative thereof, an antibody or antigen binding fragment or derivative thereof, a T-cell receptor or any antigen binding fragment or derivative thereof, an aptamer, nanobody, or other molecule antigen binding construct specifically binding to the EV surface marker. A binding agent according to the invention refers to any agent that specifically and/or (preferably) selectively binds to its respective indicated target. In addition to the aforementioned agents, such binding agents can be selected from small or large molecules or molecule complexes used in affinity labelling, any staining agents or derivatives thereof and biological cells, if they bind to respective EV surface structures, for example via their receptors. These binding agents may be situated in solution, dispersion or fixated on a surface, for example on the surface of a well or on the surface of nano/microstructures or particles. In preferred embodiments of the invention, the binding-agent is an antibody or antigen binding fragment or derivative thereof.

In preferred embodiments of the invention, the binding-agent comprises a readout tag, such as a readout tag capable of eliciting a detectable signal. A readout tag in the context of the invention comprises a functional moiety that is chemically or physically attached to the binding-agent. This functional moiety may interact with its environment, for example by absorption or emission of electromagnetic waves, magnetism, enzymatic functions, or feature biological activity. Alternatively, the readout tag is detectable by being a sequence with defined length and/or arrangement of individual molecular or atomic constituents. In case of molecular constituents, this comprises amino acid or nucleic acid sequences or polymer chains. In case of atomic constituents chains of individual atoms such as an oligo (or poly) sulfide chains might be used as a readout tag. In sufficient concentration, these tags are used to detect, preferably quantify the presence of the binding-agent. In preferred embodiments of the invention, the readout tag is a fluorescent tag.

In a preferred embodiment, detecting the (specific) binding of the binding-agent to the EV surface marker is based on an immunological-, spectroscopic-, and/or spectrometric method. In another preferred embodiment of the invention, the EV surface marker is detected by an immunological-, spectroscopic-, and/or spectrometric method. In a preferred embodiment, the spectrometric method is conducted with a variant of matrix-assisted laser desorption/ionization (MALDI) and/or electrospray ionization (ESI). Preferably, these spectrometric methods can be used for confirming and/or quantifying the presence or absence of EV surface markers with or without their prior labelling using a binding-agent. Spectrometric techniques in the context of the invention may be chosen from any mass spectrometry technique that is state of the art such as coupled gas chromatography-mass spectrometry (GC-MS), liquid chromatography-mass spectrometry (LC-MS) and/or capillary electrophoresis-mass spectrometry (CE-MS). This also includes ionization techniques such as MALDI or ESI or other variants of mass spectrometry setups. This further includes a combination with other analysis techniques such as with other mass-based detection methods, for example surface plasmon resonance (SPR) spectroscopy. Mass spectrometric methods may be performed on EV-preparations directly, or on samples that contain EV-fragments. Such samples are EV-preparations that have been subject to digestion solutions. If a mass-spectrometry method is used to detect binding agents, these can optionally be labelled. Any commonly used labels such as isobaric tags, ¹⁸O labels, dimethyl labels and/or isotope-coded affinity labels may be used.

In preferred embodiments, the spectroscopic method comprises UV/Vis spectroscopy, fluorescence spectroscopy, nuclear magnetic resonance (NMR)-spectroscopy, and/or infrared (IR)-spectroscopy, Raman-spectroscopy and/or surface plasmon resonance spectroscopy. This includes any variations of such setup and setups that rely on the physical principles of these methods. For example, this comprises combinatory setups that combine spectroscopy with microscopy such as in fluorescence microscopy, IR microscopy or Raman microscopy. It also includes combinations with biological/immunological assays that rely on a spectroscopic readout. In case of spectroscopic methods that are based on SPR, NMR, IR and Raman spectroscopy, EV surface markers may also be detected without prior labelling.

In preferred embodiments, the immunological method comprises Enzyme-linked Immunosorbent Assay (ELISA), enzyme immunoassay (EIA), fluorescence immunoassay (FIA), chemiluminescence immunoassay (CIA), radioimmunoassay (RIA), Western-Blot and/or peptide-array. Any immunological/biological/in vitro assay that is suitable for detecting EV surface markers or EV membrane molecules is included herein. In these assays, binding agents that bind to EV surface markers may be fixated on surfaces, such as the surface of a microscopy slide or the surface of a well plate. These immunological methods can be used in conjunction with other methods and setups that are for example based on spectroscopy or microscopy. Preferably, the binding-agents of these immunological methods directly give a detectable signal, such as in the case of a tag comprising fluorescent moieties. It is also possible, that the binding-agent merely fixates the EV, EV fragment or EV surface marker on a surface, depending on the presence of an EV surface marker. Other agents that are able to specifically or non-specifically bind to the EV, EV fragments, or EV surface markers and that are capable of eliciting a signal for readout can additionally bind to the fixated species. In other embodiments the binding agents or their tags may act as intermediates or precursors for other binding agents to specifically bind to, for example in the case of biotin tagging systems or enzyme tagging systems. In other embodiments, binding agents may comprise tags that can be detected by their catalytic function, such as horseradish peroxidase, alkaline phosphatase, glucose oxidase and β-galactosidase.

In preferred embodiments of the invention, the presence or absence of the EV surface marker is compared to the presence or absence of the EV surface marker of a control or reference. Preferably, this control or reference relates the presence or absence of the EV surface marker to a known EV and/or surface marker concentration, and/or a known therapeutic efficacy.

In preferred embodiments of the invention, the reference is any of the following:
(i) an EV-reference-preparation comprising predetermined amount of EV surface marker, optionally wherein the EV reference is not comprising any of the EV surface marker.
(ii) an EV-reference-preparation comprising predetermined amount of EV surface marker that is associated with the binding-agent of any of the prior embodiments;
(iii) an EV-reference-preparation with a predetermined therapeutic efficacy, optionally wherein the EV-reference is not comprising any therapeutic efficacy.

Preferably, the reference is suitable for any of the aforementioned detection methods. Preferably, the reference comprises any of the abovementioned surface-marker binding agents and various variants of their tags. Furthermore, the reference may be provided in form a kit that further comprises instructions on how to perform the detection of EV surface structures, and chemicals that are needed for such an assay.

In preferred embodiments of the invention, the predetermined therapeutic efficacy is determined by an immunomodulatory assay. Preferably, such an immunomodulatory assay is chosen from any assay that is suitable for determining immunosuppression. Such assays comprise on measuring leukocyte, preferably lymphocyte, proliferation, concentration and function, antibody production, cytotoxic lymphocyte function, NK cell function, dendritic cell function, cytokine release, protein array expression, or any other parameter that indicative of immunosuppressive efficacy. Preferably, the immunomodulatory assay is a mixed lymphocyte reaction assay (MLR). For example, such a mixed lymphocyte reaction assay can be based on measuring the proliferation of CD4+ T-Cells and/or CD4+CD25+CD54+ T-cells when treated with EVs with a predetermined amount of EV surface marker. Most preferably, this mixed lymphocyte reaction assay is the assay disclosed in WO 2014/013029.

In a preferred embodiment of the invention, the EV surface marker is a surface protein, preferably wherein the EV surface marker is CD81, and/or CTLA-4. CD81 is also known under the following names: 26 kDa cell surface protein, Teraspanin-28 (Tspan-28) or Target of the Antiproliferative Antibody 1 (TAPA-1). With exception to erythrocytes, blood platelets and neutrophils, the cell surface protein CD81 can be found on the surface of cells with hematopoietic origin. It belongs to the tetraspanin superfamily, and has been identified as a component of the B lymphocyte receptor. CD81 associates with various immune molecules on T and B lymphocytes as well as other cell types to facilitate cell-to-cell communication at the immune synapse interface between antigen-presenting cells (APCs) and T lymphocytes. Furthermore, CD81 has also been shown to regulate cell migration. In the context of the invention, the term CD81 refers to any of the isoforms of human CD81 that are reported on uniprot.org as of date (15.04.2021). This includes one canonically described isoform (SEQ ID NO: 1) and ten potential isoforms that are computationally mapped (SEQ ID NO: 2-11).

In a preferred embodiment of the invention, the EV surface marker is CTLA-4. The term CTLA-4 may refer to any isoform of the protein CTLA-4, also known as CD152 or Cytotoxic T-lymphocyte protein 4. It is an inhibitory receptor that is acting as a negative regulator of T-cell responses when it is bound by its natural B7 family ligands CD80 and CD86. As of 19.07.21, five different CTLA-4 isoforms are reported (https://www.uniprot.org/uniprot/P16410), which are generated by alternative splicing. (SEQ ID NO: 12-16). Isoform 1 (SEQ ID NO 12) corresponds to the canonical sequence of CTLA-4. It exhibits four exons that code for a signal sequence (leader peptide, positions 1-35), ligand binding domain (positions 36-161), transmembrane domain (positions 162-182) and cytoplasmic tail (positions 183-223). Isoform 2 (SEQ ID NO 13) is also known as SS-CTLA-4. Isoform 5 (SEQ ID NO 16), commonly referred to as sCTLA-4, is a functional molecule with specific CD80 and CD86 binding ability that is generated by alternatively spliced CTLA-4 mRNA. The mRNA encoding sCTLA-4 consists of three exons: exon 1 encoding the leader peptide, exon 2 the ligand-binding domain, and exon 4 the cytoplasmic tail, but it lacks exon 3 encoding the transmembrane domain. The spliced transcript produces a 23-kDa sCTLA-4 that is characterized by a cytoplasmic tail shorter than that of the full-length form of the CTLA-4 antigen. As sCTLA-4 lacks the cysteine residue at position 120, it is expressed as a monomer. In addition, sCTLA-4 contains the MYPPPY motif located in the extracellular domain which is critical for B7 molecule binding. Therefore it maintains the ability to bind CD80/CD86 and to participate in the B7/CTLA-4/CD28 signaling pathway of T-cell regulation. However, little is known of any regulatory role for natural sCTLA-4.

A second aspect of the invention pertains to a method for producing an Extracellular Vesicle (EV) or EV-preparation with therapeutically sufficient efficacy, comprising the following steps:
(i) Providing a secretory cell;
(ii) Culturing the secretory cell in a culture medium (and under conditions) that allows for a secretion of EVs into the culture medium;
(iii) Harvesting of the secreted EVs to obtain a harvested EV or EV-preparation;
(iv) Performing the method according to the first aspect of the invention with the harvested EV or EV-preparation to determine its therapeutic efficacy;
(v) Selecting the harvested EV or EV-preparation with therapeutically sufficient efficacy.

Any method that is suitable for separating EVs from culture medium can be used for conducting step (iii). Preferably, EVs are harvested using a method selected from the group consisting of ultracentrifugation based methods such as differential centrifugation, density gradient centrifugation, isopycnic ultracentrifugation, or moving-zone ultracentrifugation, immunoaffinity-based methods that rely for example on antibody-coated magnetic beads, exosome precipitation using reagents such as PEG or Exoquick™, chromatography based methods such as anion-exchange chromatography or size exclusion chromatography, ultrafiltration-methods, flow field-flow fractionation, microfluidic devices, membrane-based methods, fluorescence-assisted cell sorting, tangential flow filtration, and/or methods that are rely on on-chip isolation devices such as an ExoTIC. In most preferred embodiments of the invention, harvesting of the EV or EV-preparation is conducted by ultracentrifugation, size-based fractionation, anion-exchange chromatography and/or PEG-precipitation.

Herein, the selection of the harvested EV or EV-preparation with therapeutically sufficient efficacy of step (v) is based on the determined therapeutic efficacy according to any prior embodiment of the invention. This can be based on the mere presence or absence of a pharmacological effect or on meeting a preferred ratio of therapeutic efficacy of the EV-preparation.

In a preferred embodiment, the secretory cell is a mammalian cell, preferably a human cell. The secretory cell can be chosen from a list consisting of adipocytes, adult and neonatal stem cells, astrocytes, B-cells, cardiomyocytes, chondrocytes, cornea epithelial cells, dendritic cells, endocrine cells, endothelial cells, epithelial cells, fibroblasts, glia cells, granulocytes, hematopoietic cells, hematopoietic stem cells, hepatocytes, keratinocytes, intestinal epithelial cells, liver cells, lung epithelial cells type I, lung epithelial cells type II, lymphocytes, macrophages, mammary epithelial cells, melanocytes, mesangial cells, mesenchymal stem/stromal cells, muscle cells, myoblast, natural killer cells, neuronal cells, neuronal stem cells, neutrophiles, osteoblasts, pancreatic beta cells, pericytes, preadipocytes, progenitor cells, prostate epithelial cells, renal epithelial cells, renal proximal tubule cells, retinal pigment epithelial cells, Sertoli cells, skeletal muscle cells, smooth muscle cells, stem cells, stroma cells, T-cells and subsets of said cell types. Said secretory mammalian cell can originate from any mammalian organism, for example mice, rats, monkeys, pigs, dogs, cats, cows, sheep, goats. Preferably, it is a human cell.

In another preferred embodiment, the secretory cell is a Mesenchymal Stromal Cell (MSC). In the present disclosure, the term “mesenchymal stromal cell” is a multipotent stromal cell that can differentiate into a variety of cell types, including, but not limited to osteoblasts (bone cells), chondrocytes (cartilage cells), myocytes (muscle cells) and adipocytes (fat cells). These cells are also known as “mesenchymal stem cells” due to their multipotency. Initially, the therapeutic function of MSCs was attributed to this cellular differentiation potential. According to newer literature, the effects of MSCs, such as the suppression of immune responses, are mediated mainly by paracrine factors, for example by the secretion of via MSC-EVs. MSC-EVs can be identified by the presence of the surface markers CD73, CD90, CD105 and the absence of CD14, CD34, and CD11b. The MSCs that are used in the present invention comprise primary MSCs and modified MSCs that originate from biological sources comprising bone marrow, adipose tissue, umbilical cord blood, umbilical cord tissue, placenta tissue, periodontal ligament, trabecular bone, synovial membrane, periosteum, muscle and skin tissue.

A third aspect of the invention pertains to a method for producing a therapeutically active Extracellular Vesicle (EV) or EV-preparation with therapeutically sufficient efficacy, comprising the following steps:
(i) Providing a secretory cell;
(ii) Enhancing in the secretory cell the secretion of therapeutically efficient EVs;
(iii) Culturing the secretory cell in a culture medium (and under conditions) that allows for a secretion of EVs into the culture medium
(iv) Harvesting of the secreted EVs to obtain a harvested EV or EV-preparation;
(v) Performing the method according to the first aspect of the invention with the harvested EV or EV-preparation to determine its therapeutic efficacy;
(vi) Selecting the harvested EV or EV-preparation with therapeutically sufficient efficacy.

In preferred embodiments of the invention, enhancing in the secretory cell the secretion of therapeutically efficient EVs comprises increasing the amount of secreted EVs provided by the secretory cell and/or increasing the amount of surface marker in the EV or EV-cell preparation. Preferably, this is achieved with methods based on hypoxic and/or inflammatory priming of the secretory cells. In a preferred embodiment, enhancing in the secretory cell the secretion of therapeutically efficient EVs is performed under normoxic conditions or hypoxic conditions. In a preferred embodiment, culturing the secretory cell in a culture medium (and under conditions) that allows for a secretion of EVs into the culture medium is performed under normoxic conditions or hypoxic conditions. Normoxic conditions in the context of the invention refer to any cell culture environment that features an oxygen concentration in the medium that is in equilibrium with 21% dissolved oxygen in the gaseous phase of the headspace of an incubation reactor at 37° C. Hypoxic conditions refer to any cell culture environment in which the oxygen concentration of the medium is in equilibrium with 1-15%, preferably 1-10% dissolved oxygen in the gaseous phase of the headspace of an incubation reactor at 37° C. In other preferred embodiments, enhancing in the secretory cell the secretion of therapeutically efficient EVs is performed by contacting the secretory cell with an immunomodulatory binding-agent, for example by adding the immunomodulatory binding-agent to the culture medium. The immunomodulatory binding agent can be chosen from the group consisting of TGF-β, TNF-α, IFN-γ, lipopolysaccharide (LPS), IL-1α, IL-1β, and Toll like receptor (TLR) agonists. Other preferred strategies to improve the performance of secreted EVs in the context of the present invention are based on altering the EV cargo composition. This may be achieved by overexpression of the GATA-4 transcription factor (improved cardiac function), mutations in the HIF-1α gene (to an oxygen-resistant form), the overexpression of Akt, the transfection of tumour necrosis factor-related apoptosis-inducing ligand (TRAIL) in the secretory cell or the engineering of the secretory cell to express a variety of miRNAs.

In preferred embodiments the invention pertains to an Extracellular Vesicles (EV) preparation obtainable by any one of the previous aspects of the invention.

In a fourth aspect, the invention pertains to an Extracellular Vesicles (EV) preparation obtainable by the method of the second and third aspect of the invention.

In a fifth aspect, the invention pertains to a use of the Extracellular Vesicles (EV) preparation of the previous aspects of the invention, in the manufacturing of a medicament. In preferred embodiment of the invention, the medicament comprises at least one EV. The EV according to the present invention can function as an active agent in the medicament, or can function as a carrier for other drugs. Preferably, the EV functions as the active agent.

In more preferred embodiments of the invention, the medicament comprises the EV-preparation of the fourth aspect of the invention. Preferably, the medicament additionally comprises further active ingredients and carriers. The carrier may be physiologically acceptable, for example, pharmaceutically or cosmetically acceptable. The carrier may include saline, sterile water, Ringer's solution, buffer, cyclodextrin, a dextrose solution, a maltodextrin solution, glycerol, ethanol, liposomes, or a combination thereof, which are generally used. In addition, the carrier may include an antioxidant, a diluent, a dispersant, a surfactant, a binder, a lubricant, or a combination thereof. In preferred embodiments of the invention, the manufacturing of the medicament comprises performing any of the methods of the first three aspects of the invention.

Preferably, the medicament is administered by a route selected from intratumoral, intraperitoneal or intravenous administration. The medicaments and compositions may be formulated in fluid or solid form. Fluid formulations may be formulated for administration by injection to a selected region of the human or animal body.

In preferred embodiments of the invention, the medicament is used as a complementing and/or alternative and/or second-line therapy to a steroid-based therapy. In preferred embodiments of the invention, the medicament is used for treatment of an immune mediated, endocrine, orthopaedic, neurodegenerative, cardiovascular and/or respiratory disease or injury.

In preferred embodiments of the invention, the medicament is used for the treatment of a disease or injury of the liver, bone marrow, lung, spleen, brain, pancreas, stomach or intestine. In preferred embodiment of the invention, the medicament is used for immunomodulatory/immunoregulatory, anti-inflammatory, anti-fibrotic, angiogenetic, and/or regenerative treatment of an injury or a disease.

In the following, special aspects and embodiments of the invention are provided. Preferably, the above aspects, embodiments and definitions apply and/or may be combined with the special preferred aspects, embodiments and/or definitions provided below.

In special preferred embodiments, the EV surface marker is selected from the group of CTLA-4, PD1, PD-L1 and PD-L2. In further special preferred embodiments, the EV surface marker is CTLA-4, PD-L1, PD-L2 and/or combinations thereof. Preferably, the EV surface marker is selected from the group consisting of CTLA-4, PD1, PD-L1 and PD-L2. Preferably, the EV surface marker is CTLA-4 and/or PD-L1. Preferably, the EV surface marker is CTLA-4. Preferably the EV-surface marker is PD-L1. Preferably, the EV surface marker is PD-L2. In special preferred embodiments, the EV surface marker is at least one EV surface marker, preferably at least two EV surface markers, more preferably at least three EV surface markers.

In special preferred embodiments, providing the EV or EV preparation does not comprise a treatment by surgery. For example, the EV or EV preparation may be manufactured from a cell culture.

In special preferred embodiments, the method for assessing the therapeutic efficacy of an Extracellular Vesicle (EV) or an EV-preparation, comprises the following steps:
(i) Providing the EV or EV-preparation;
(ii) Detecting, optionally by using a binding-agent, the presence or absence of at least one EV surface marker, preferably wherein the at least one EV surface marker is selected from the group of CTLA 4, PD1, PD-L1 and PD-L2, wherein the EV surface marker is associated with the EV or EV-preparation;
Wherein the presence of the at least one EV surface marker associated with the EV or EV-preparation is indicative of a therapeutically sufficient efficacy of the EV or EV-preparation.

In special preferred embodiments, the terms “detect” or “detection” refer to a “quantification”. In special preferred embodiments, the term “presence” refers to a quantity, for example a concentration. Preferably, the quantity surpassing the threshold is indicative of a therapeutically sufficient efficacy of the EV or EV-preparation.

In special preferred embodiments of the invention, detecting the presence or absence of the EV surface maker in step ii) comprises detecting a (specific) binding between the binding-agent and the EV surface marker. Preferably, detecting the (specific) binding of the binding-agent to the EV surface marker is based on an immunological-, spectroscopic-, and/or spectrometric method. In another preferred embodiment of the invention, the EV surface marker is detected by an immunological-, spectroscopic-, and/or spectrometric method. In special preferred embodiments, the spectrometric method is conducted with matrix-assisted laser desorption/ionization (MALDI) and/or electrospray ionization (ESI) or a variant thereof.

In special preferred embodiments, the EV is characterized by being an EV secreted from a secretory cell. The secretory cell may be a cell secretory cell of any aspect of the invention. Preferably, the secretory cell is a mammalian cell. Preferably, the secretory cell is a human cell. In special preferred embodiments, the secretory cell is an MSC. In special preferred embodiments, the EV is secreted from an MSC. The MSC may be an MSC according to any aspect of the invention. In special preferred embodiments, the MSC-EVs are characterized by the presence of the surface markers CD73, CD59, CD64 and CD81.

In special preferred embodiments, the secretory cell comprises a nucleic acid which encodes an EV surface marker. In special preferred embodiments of the invention, the term “nucleic acid” includes “polynucleotide”, “oligonucleotide”, and “nucleic acid molecule”, and generally refers to a polymer of DNA or RNA, which can be single-stranded or double-stranded, synthesized or obtained (e.g. isolated and/or purified) from natural sources, which can contain natural, non-natural or altered nucleotides, and can contain a natural, non-natural or altered internucleotide linkage, such as a phosphoroamidate linkage or a phosphorothioate linkage, instead of the phosphodiester found between the nucleotides of an unmodified oligonucleotide.

In special preferred embodiments, the secretory cell is characterized by an expression, preferably an overexpression of the EV surface marker. In this context, overexpression preferably refers to an increased expression of the EV surface marker in the secretory cell when compared to a wild-type secretory cell.

In special preferred embodiments, the secretory cell is characterized by a native expression and/or is capable of a native expression of the EV surface marker. Without wishing to be bound by theory, the term “native expression” preferably refers to the feature that the secretory cell comprises a nucleic acid sequence encoding the EV surface marker that was not artificially, meaning by human intervention (for example by the use of a vector), introduced into the secretory cell. The term “capable of a native expression of the EV surface marker” preferably refers to the feature that the secretory cell comprises a nucleic acid sequence encoding the EV surface marker that was not artificially introduced into the secretory cell and the secretory cell may be in a state, in which the EV surface marker is not expressed. The expression of the EV surface marker in such a secretory cell may be inducible, for example by exposing the cell to a stimulus. In preferred embodiments of the invention, the EV surface marker is native protein of the secretory cell.

In special preferred embodiments of the invention, the term nucleic acid may refer to a recombinant nucleic acid. Preferably, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. In special preferred embodiments the replication may be in vitro replication or in vivo replication.

In special preferred embodiments, the invention pertains to a vector comprising a nucleic acid of the invention. Preferably, the vector is an expression vector or a recombinant expression vector. Preferably, the term “recombinant expression vector” refers in context of the present invention to a nucleic acid construct that allows for the expression of an mRNA, protein or polypeptide in a suitable host cell, such as the secretory cell. The recombinant expression vector of the invention can be any suitable recombinant expression vector, and can be used to transform or transfect any suitable host. Suitable vectors may include those designed for propagation and expansion or for expression or both, such as plasmids and viruses. Preferably, the recombinant expression vector is a viral vector, e.g., a retroviral vector. In special preferred embodiments, the retroviral vector is a lentiviral vector. Such a lentiviral vector may be used to for the delivery and expression of a nucleic acid according to the invention to both dividing and non-dividing cell populations in vitro and in vivo. Preferably, lentiviral vectors include those based on HIV, FIV and EIAV. See, e.g., Lois, C, et al., Science, 2002, 295 (5556): p. 868-72.

In the context of special embodiments of the invention, the recombinant expression vector preferably comprises regulatory sequences, such as transcription and translation initiation and termination codons, which are specific to the type of secretory cell (e.g., bacterium, fungus, plant, or animal), into which the vector is to be introduced and in which the expression of the nucleic acid encoding the EV-surface marker may be performed. Furthermore, the vector of the invention may include one or more marker genes, which allow for selection of transformed or transfected secretory cells.

In special preferred embodiments, the recombinant expression vector comprises a native or normative promoter operably linked to the nucleotide sequence encoding the nucleic constructs of the invention, or to the nucleotide sequence, which is complementary to or which hybridizes to the nucleotide sequence encoding the constructs of the invention. The selections of promoters may include, e.g., strong, weak, inducible, tissue-specific and developmental-specific promoters. The promoter can be a non-viral promoter or a viral promoter. The inventive recombinant expression vectors can be designed for either transient expression, for stable expression, or for both. Also, the recombinant expression vectors can be made for constitutive expression or for inducible expression. Preferably, the lentiviral vector of the present invention regulates the expression of a nucleic acid of the invention by a promoter. Preferably, the promoter is selected from the group of cytomegalovirus (CMV), respiratory syncytical virus (RSV), human elongation factor-1 alpha (EF-1α) and tetracycline response elements (TRE) promoter. In these special preferred embodiments, the promoter is operably linked to the nucleic acid encoding the EV-biomarker.

In special preferred embodiments, the EV the secretory cell is characterized by a recombinant expression of the EV surface marker and/or is capable of a recombinant expression the EV surface marker. The term “capable of a recombinant expression of the EV surface marker” preferably refers to the feature that the secretory cell comprises a recombinant nucleic acid encoding the EV surface marker and the secretory cell may be in a state, in which the EV surface marker is not expressed. The expression of the EV surface marker in such a secretory cell is preferably inducible, for example by exposing the cell to a stimulus.

In special preferred embodiments, the nucleic acid sequence encodes for an EV surface marker or functional variant thereof. Preferably, the nucleic acid sequence comprises or consists of a sequence having at least 50%, 60%, 70%, 80%, 90%, 95%, 98%, 99%, or preferably 100% sequence identity to an amino acid sequence selected from SEQ ID NOS: 1 to 25. Preferably, the nucleic acid sequence comprises or consists of a sequence having at least 50%, 60%, 70%, 80%, 90%, 95%, 98%, 99%, or preferably 100% sequence identity to an amino acid sequence selected from SEQ ID NOS: 1 to 11. Preferably, the nucleic acid sequence comprises or consists of a sequence having at least 50%, 60%, 70%, 80%, 90%, 95%, 98%, 99%, or preferably 100% sequence identity to an amino acid sequence selected from SEQ ID NOs: 12 to 16. Preferably, the nucleic acid sequence comprises or consists of sequence having at least 50%, 60%, 70%, 80%, 90%, 95%, 98%, 99%, or preferably 100% sequence identity to an amino acid sequence selected from SEQ ID NOs: 17 to 19. Preferably, the nucleic acid sequence comprises or consists of a sequence having at least 50%, 60%, 70%, 80%, 90%, 95%, 98%, 99%, or preferably 100% sequence identity to an amino acid sequence selected from SEQ ID NOS: 19 to 22. Preferably, the nucleic acid sequence comprises or consists of a sequence having at least 50%, 60%, 70%, 80%, 90%, 95%, 98%, 99%, or preferably 100% sequence identity to an amino acid sequence selected from SEQ ID NOs: 22 to 25.

In a special sixth aspect, the invention pertains to a method for manufacturing an Extracellular Vesicle (EV) or EV-preparation with therapeutically sufficient efficacy, comprising the following steps:
(i) Providing a secretory cell;
(ii) Culturing the secretory cell in a culture medium that allows for the secretion of an EV into the culture medium;
(iii) Sorting the secreted EV into at least two fractions, depending on the presence or absence of an EV surface marker that is associated with the EV;
Wherein a fraction of the at least two fractions is the Extracellular Vesicle (EV) or EV-preparation with therapeutically sufficient efficacy.
In special preferred embodiments, the fraction of the at least two fractions comprises the secreted EV and comprises a presence of the EV surface marker.

In special preferred embodiments, the secretory cell is a secretory cell of any aspect of the invention. In special preferred embodiments, the EV surface marker is a surface marker according to any aspect of the invention. In special preferred embodiments, the culturing the secretory cell is conducted according to the culturing of the second and third aspect of the invention.

In special preferred embodiments, the method of manufacturing comprises an additional step of enhancing in the secretory cell the secretion of therapeutically efficient EVs. Preferably, said step of enhancing is conducted simultaneously or after step (ii) “Culturing the secretory cell in a culture medium that allows for the secretion of an EV into the culture medium”.

In special preferred embodiments, the sorting comprises performing an affinity chromatography. Preferably, the affinity chromatography comprises the use of a stationary phase with a surface comprising the binding agent of the other aspects of the invention.

In special preferred embodiments, the method of producing an EV or EV-preparation and/or the method of manufacturing an EV or EV-preparation may comprise a step of genetically modifying the secretory cell to express, preferably recombinantly express, the EV surface marker. Preferably, the genetic modification comprises introducing the nucleic acid and/or the vector according to the invention into the secretory cell, for example by transduction and/or transfection. Preferably the genetic modification comprises bringing into contact the secretory cell with the nucleic acid and/or the vector according to the invention.

In special preferred embodiments, the method comprises an additional step of isolating the EV that is conducted after step (ii) and/or after step (iii). For this step, any method that might be used in the harvesting step according to the second or third aspect of the invention might be used for isolating the EV.

In a special seventh aspect, the invention pertains to an Extracellular Vesicle (EV) preparation with therapeutically sufficient efficacy obtainable by the methods of the first, second, third, and/or sixth aspect of the invention.

In a special eighth aspect, the invention pertains to a use of the Extracellular Vesicle (EV) preparation of any other aspects of the invention, in the manufacturing of a medicament. In special preferred embodiments, the use comprises performing the method of any other aspect of the invention.

In a special ninth aspect, the invention pertains to an EV or EV preparation according to any other aspect of the invention and/or a medicament according to any other aspect of the invention for use in the treatment of a condition or disease. Preferably, the EV preparation is the medicament according to the other aspects of the invention.

In special preferred embodiments the disease is an immune mediated, endocrine, orthopaedic, neurodegenerative, cardiovascular and/or respiratory disease or injury. In special preferred embodiments, of the invention, the disease is a disease or injury of the liver, bone marrow, lung, spleen, brain, pancreas, stomach and/or intestine.

In special preferred embodiments, the disease is an inflammatory disease, preferably, wherein the inflammatory disease is an allergy, asthma, an autoimmune disease, coeliac disease, glomerulonephritis, hepatitis, inflammatory bowel disease, reperfusion injury and/or Graft-versus-Host disease. Preferably, the disease is Graft-versus-Host disease.

In special preferred embodiments, the disease is a T cell mediated disease. Preferably the T cell mediated disease is associated with increased, and/or preferably aberrant, CD4 and/or CD8 T cell activity. Especially preferred examples for such a T cell mediated disease are rheumatoid arthritis, diabetes mellitus type 1, MHC-associated inflammatory eye disease, multiple sclerosis, celiac disease, autoimmune encephalomyelitis, Rasmussen's encephalitis, and Paraneoplastic syndromes.

In a special tenth aspect, the invention pertains to a method for the treatment of a condition or disease, the method comprising administering to a subject in need thereof an EV, EV preparation and/or medicament according to any other aspect of the invention. Preferably, the condition or disease is a condition or disease according to any other aspect of the invention.

In special preferred embodiments, the treatment comprises preventing the condition or disease. In this context, preventing of the condition or disease may refer to impeding or delaying the onset and/or slowing down the progression of the condition or disease.

In special preferred embodiments, the treatment comprises administering to the subject a therapeutic dose of the EV, the EV preparation and/or the medicament. Preferably, the therapeutic dose is a dose that alleviates a parameter associated with the condition or disease. Preferably, the parameter associated with the condition or disease is the presence, such as a concentration, of an inflammatory molecule and/or an activity of an immune cell, preferably, wherein the immune cell is a T cell. The therapeutic dose may depend on the subject (such as the severity of the condition or disease of the subject, weight and/or sex of the subject), the type of the disease, and the route of administration. In special preferred embodiments, the therapeutic dose comprises an EV concentration of 1.5×10⁸-1.5×10¹³ EV/mL. Preferably, the therapeutic dose comprises an EV concentration of 1.5×10⁸-1.5×10⁹ EV/mL, 1.5×10⁸-1.5×10⁹ EV/mL, 1.5×10⁹-1.5×10¹⁰ EV/mL, 1.5×10¹⁰-1.5×10¹¹ EV/mL, 1.5×10¹¹-1.5×10¹² EV/mL or 1.5×10¹²-1.5×10¹³ EV/mL. A person skilled in the art will be able to select and/or determine a suitable concentration and treatment regimen. Preferably, the treatment comprises a single administration of a therapeutic dose. In special preferred embodiments, the treatment comprises multiple administrations of the EV, the EV preparation and/or the medicament, preferably with a short time period in-between each administration (such as a time period of 12 h, 1 day, 2 days or 3 days in-between each administration).

In special preferred embodiments, the secretory cell is a cell that is autologous to the subject. Preferably, the secretory cell, preferably the secretory cell that is autologous to the subject, is genetically modified to express an EV surface marker according to the present invention.

In special preferred embodiments, administration of the EV, the EV preparation and/or the medicament is conducted systemically and/or directly to an area to be treated. Preferably, the systemic administration is conducted parenterally such as by intravenous, intraarterial, or intraperitoneal administration. Preferably, the administration to an area to be treated is conducted by applying the EV, the EV preparation and/or the medicament topically and/or by direct injection into an organ or tissue in need thereof.

In a special preferred embodiment, the EV surface marker is PD-L1. The term PD-L1 may refer to any isoform of the protein PD-L1, also known as PDCD1 ligand 1, programmed death ligand 1 and hPD-L1. It is a ligand for the inhibitory receptor PD1, modulates the activation threshold of T cells and limits T cell effector response. As of date, three different PD-L1 isoforms are reported (https://www.uniprot.org/uniprotkb/Q9NZQ7/entry), which are generated by alternative splicing. (SEQ ID NO: 17-19). Isoform PD-L1I (SEQ ID NO: 17) corresponds to the canonical sequence of PD-L1. The protein exhibits an extracellular topological domain (positions 19-238), a helical transmembrane domain (positions 239-259) and a cytoplasmic topological domain (positions 260-290). Furthermore, at positions 19-127, the protein comprises an Ig-like V-type domain, and at positions 133-225, the protein comprises a Ig-like C2 type domain. Isoform PD-L1II (SEQ ID NO: 18) is missing the Ig-like V-type domain. Isoform PD-L1III (SEQ ID NO: 19) may be produced at very low levels due to a premature stop codon in the mRNA.

In a special preferred embodiment, the EV surface marker is PD-L2. The term PD-L2 may refer to any isoform of the protein PD-L2, also known as PD-1 ligand 2, PD-L2, PDCD1 ligand 2 and programmed death ligand 2. It is involved in T cell proliferation and IFNG production in a PDCD1-independent manner. As of date, three different PD-L2 isoforms are reported (https://www.uniprot.org/uniprotkb/Q9BQ51/entry), which are generated by alternative splicing. (SEQ ID NO: 20-22). The isoform PD-L2I (SEQ ID NO: 20) corresponds to the canonical isoform of PD-L2. The protein comprises an extracellular topological domain (positions 20-220), a helical transmembrane domain (positions 221-241), and a cytoplasmic topological domain (positions 242-273). Furthermore, it comprises an Ig-like V-type domain at positions 21-118 and an Ig-like C2 domain at positions 122-203. The isoform PD-L2II (SEQ ID NO: 21) is missing the Ig constant-like domain. The isoform PD-L2III (SEQ ID NO: 22) is predicted to be a secreted, soluble variant.

In a special preferred embodiment, the EV surface marker is PD1. The term PD1 may refer to any isoform of the protein PD1, also known as Programmed cell death protein 1. Preferred isoforms of the protein isoforms can as of date be found on uniport (https://www.uniprot.org/uniprotkb/Q15116/entry#names_and_taxonomy). Amongst others, the protein is known as inhibitory receptor on antigen activated T-cells. The canonical isoform of PD1 is provided in SEQ ID NO: 23. Preferably PD1 is the canonical isoform or another isoform of PD1 such as the isoforms described by SEQ ID NO: 23 and/or SEQ ID NO: 25.

In yet further special preferred embodiments, the invention pertains to the following itemized embodiments:
Item 1: A method for assessing the therapeutic efficacy of an Extracellular Vesicle (EV) or an EV-preparation, comprising the following steps:
   (i) Providing the EV or EV-preparation;
   (ii) Detecting the presence or absence of an EV surface marker, preferably CTLA 4, associated with the EV or EV-preparation, optionally by using a binding agent;
   Wherein the presence of the EV surface marker associated with the EV or EV-preparation is indicative of a therapeutically sufficient efficacy of the EV or EV-preparation.
Item 2: The method of item 1, wherein detecting the presence or absence of an EV surface maker in step ii) comprises detecting a (specific) binding between the binding-agent and the EV surface marker, preferably wherein detecting the (specific) binding of the binding agent to the EV surface marker is based on an immunological-, spectroscopic-, and/or spectrometric method, preferably wherein the spectroscopic method comprises UV/Vis spectroscopy, fluorescence spectroscopy, Nuclear magnetic resonance NMR-spectroscopy, Infrared (IR), Raman-spectroscopy and/or surface plasmon resonance spectroscopy, preferably the immunological method comprises (Enzyme-linked Immunosorbent Assay) ELISA, Enzyme Immunoassay (EIA), Fluorescence Immunoassay (FIA), Chemiluminescence immunoassay (CIA), Radioimmunoassay (RIA), Western-Blot and/or Peptide-Array, preferably wherein the spectrometric method is conducted with a variant of, matrix-assisted laser desorption/ionization (MALDI) and/or electrospray ionization (ESI).
Item 3: The method of item 1 or 2, wherein the presence or absence of the EV surface marker is compared to the presence or absence of the EV surface marker of a control or reference, preferably wherein the reference is any of the following:
   (i) an EV-reference-preparation comprising predetermined amount of EV surface marker, optionally wherein the EV reference is not comprising any of the EV surface marker.
   (ii) an EV reference-preparation comprising predetermined amount of EV surface marker that is associated with the binding agent of any one of items 1 or 2;
   (iii) an EV-reference-preparation with a predetermined therapeutic efficacy, optionally wherein the EV-reference is not comprising any therapeutic efficacy;
   preferably, wherein the predetermined therapeutic efficacy is determined by an immunomodulatory assay.
Item 4: The method of any one of items 1 to 3, wherein the binding agent is an antigen binding construct, such as a protein or nucleic acid, and preferably is a ligand molecule specific for the EV surface marker or derivative thereof, an antibody or antigen binding fragment or derivative thereof, a T cell receptor or any antigen binding fragment or derivative thereof, an aptamer, nanobody, or other molecule antigen binding construct specifically binding to the EV surface marker, preferably wherein the binding agent is an antibody or antigen binding fragment or derivative thereof.
Item 5: A method for producing an Extracellular Vesicle (EV) or EV-preparation with therapeutically sufficient efficacy, comprising the following steps:
   Providing a secretory cell;
   Culturing the secretory cell in a culture medium (and under conditions) that allows for a secretion of EVs into the culture medium;
   Harvesting of the secreted EVs to obtain a harvested EV or EV-preparation, preferably wherein harvesting of the EV or EV-preparation is conducted by ultracentrifugation, size based fractionation, anion exchange chromatography and/or PEG-precipitation;
   Performing the method of any one of items 1 to 4 with the harvested EV or EV-preparation to determine its therapeutic efficacy;
   Selecting the harvested EV or EV-preparation with therapeutically sufficient efficacy;
   Wherein the method, prior to culturing the secretory cell, optionally comprises an additional step: enhancing in the secretory cell the secretion of therapeutically efficient EVs.
Item 6: The method of item 5, wherein enhancing the secretory cell secretion comprises increasing the amount of secreted EVs provided by the secretory cell and/or increasing the amount of surface marker in the EV or EV-cell preparation, and/or wherein enhancing in the secretory cell the secretion of therapeutically efficient EVs is performed by contacting the secretory cell with an immunomodulatory binding agent, for example by adding the immunomodulatory binding agent to the culture medium.
Item 7: An Extracellular Vesicles (EV) preparation obtainable by the method of any one of items 5 or 6
Item 8: A use of the Extracellular Vesicles (EV) preparation of item 7 in the manufacturing of a medicament, preferably wherein the medicament comprises at least one EV and/or wherein the medicament comprises the EV-preparation of item 7.
Item 9: The use of item 8, wherein the manufacturing of the medicament comprises performing the method of any one of items 1 to 6.
Item 10: The use of items 8 or 9, wherein the medicament is used for treatment of an immune mediated, endocrine, orthopaedic, neurodegenerative, cardiovascular and/or respiratory disease or injury, and/or wherein the medicament is used for immunomodulatory/immunoregulatory, anti-inflammatory, anti-fibrotic, angiogenetic, and/or regenerative treatment of an injury or a disease, preferably wherein the medicament is used as a complementing and/or alternative and/or second-line therapy to a steroid-based therapy.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

### The Figures Show:

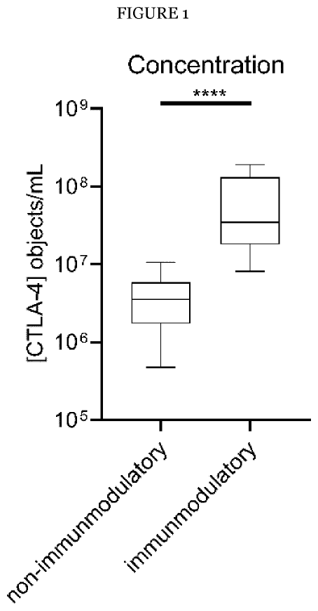

FIG. 1: Immunomodulatory active MSC-EV preparations show an increased concentration of CTLA-4 positive objects. MSC-EV preparations were anti-CTLA-4 stained and measured by IFC. A total of 32 MSC-EV preparations were measured, which were previously classified as non-immunomodulatory or immunomodulatory by the multi-donor mixed lymphocyte reaction. (non-immunomodulatory: n=16, immunomodulatory: n=16, two-tailed Mann-Whitney Test=<0.0001=****).

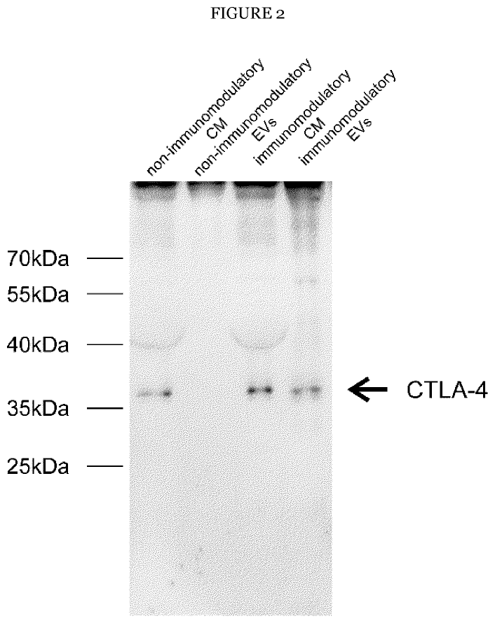

FIG. 2: Western blot confirms correlation of IFCM results. Representative Western Blots results of non-immunomodulatory and immunomodulatory MSC-EV preparation. Additionally, the corresponding condition media were applied.

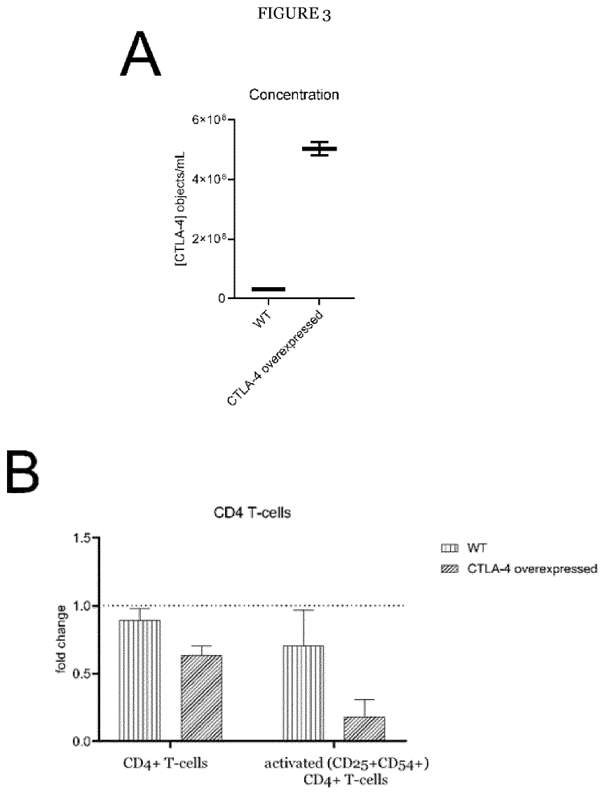

FIG. 3: EV preparations of MSCs overexpressing CTLA-4 show increased levels of CTLA-4 positive objects and enhanced immunomodulatory effects. Three independent EV preparations each of the untreated cells (wild type, WT) and the overexpressing cell line were analyzed for CTLA-4-positive objects by anti-CTLA-4 antibody and for the immunomodulatory effect by mdMLR. (FIG. 3, Panel A) Concentration measurement of CTLA-4 positive objects by IFCM of WT and CTLA-4 overexpressing (“CTLA-4 overexpressed”) EV preparations are shown as objects/mL. (FIG. 3, Panel B) Fold change of CD25 and CD54 gated living CD4+ cells of mdMLR assays cultured in the absence of any additives (pointed line), in the presence of WT MSC-EVs (“WT”) and CTLA-4 overexpressing cells (“CTLA-4 overexpressed”).

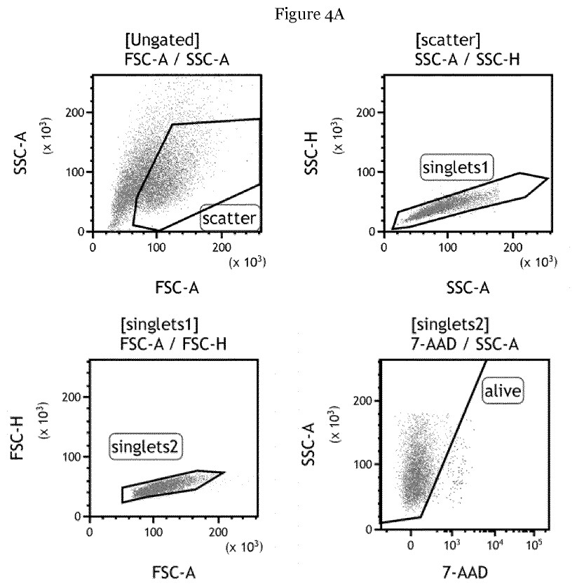

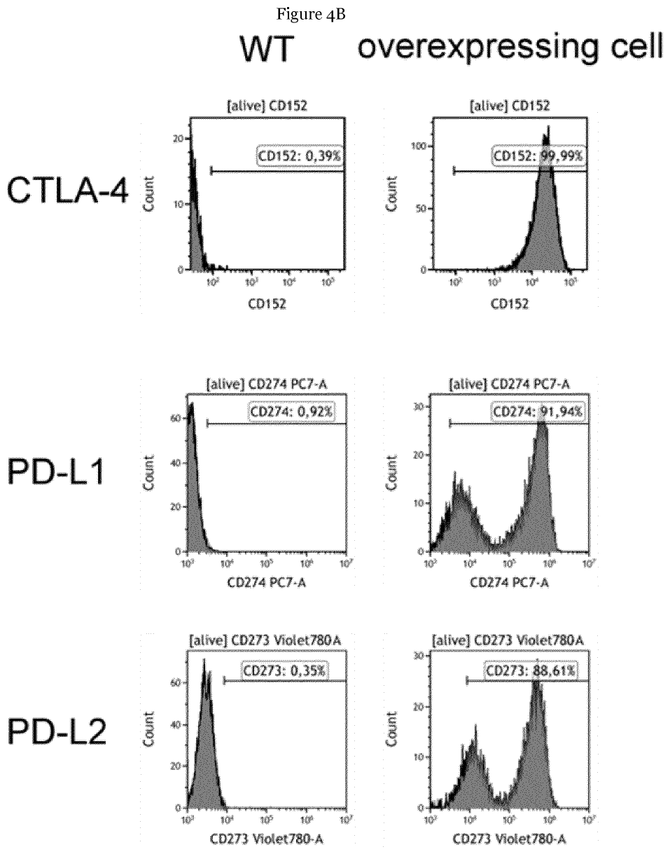

FIGS. 4A and 4B: Cell surface phenotype of expanded MSCs analyzed by flow cytometry. Cells were labelled with fluorochrome conjugated antibodies against CTLA-4, PD-L1 or PD-L2. (FIG. 4A) Gating strategy applied for MSCs. Alive MSCs were selected by using 7-AAD. (FIG. 4B) Expression levels of CTLA-4, PD-L1, and PD-L2, respectively, of the transduced MSCs.

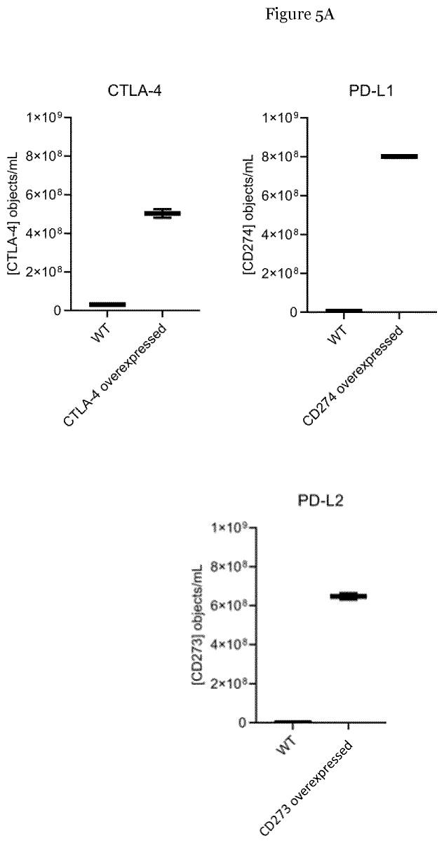

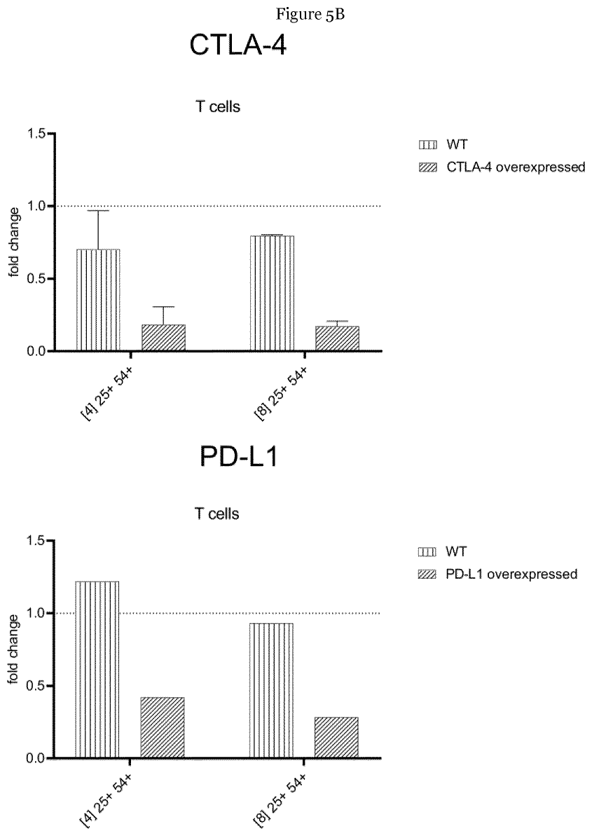

FIGS. 5A and 5B: Overexpression of CTLA-4, PD-L1, or PD-L2 results in the presence of the corresponding positive objects. MSCs were transduced to express CTLA-4, PD-L1 or PD-L2. The isolated EVs were used in mdMLR to examine immunomodulatory capacity. (FIG. 4A) Concentration measurement of CTLA-4, PD-L1 or PD-L2 positive objects by IFCM of WT and EV preparations of overexpressing cells are shown as objects/mL, respectively. (FIG. 5B) Change of CD25 and CD54 gated living CD4+ cells of mdMLR assays cultured in the absence of any additives (pointed line), in the presence of WT MSC-EVs (“WT”) and CTLA-4 and PD-L1 overexpressing cells.

The sequences show:
SEQ ID NO: 1-11: Isoforms of CD81 according to the entry of P60033 in UniProt (https://www.uniprot.org/uniprot/P60033 (16.04.2021)). The canonical isoform of CD81 is given in SEQ ID NO: 1.
SEQ ID NO: 12-16: Isoforms of CTLA-4 that are produced by alternative splicing according to the entry of P16410 in UniProt (https://www.uniprot.org/uniprot/P16410 (16.04.2021)). The canonical isoform of CTLA-4 is given in SEQ ID NO: 12.
SEQ ID NO: 17-19: Isoforms of PD-L1 that are produced by alternative splicing according to the entry of Q9NZQ7 in Uniprot (https://www.uniprot.org/uniprotkb/Q9NZQ7/entry (09.08.2022)). The canonical isoform of PD-L1 is given in SEQ ID NO: 17.
SEQ ID NO: 20-22: Isoforms of PD-L2 that are produced by alternative splicing according to the entry of Q9BQ51 in Uniprot (https://www.uniprot.org/uniprotkb/Q9BQ51/entry (09.08.2022)). The canonical isoform of PD-L2 is given in SEQ ID NO: 20.
SEQ ID NO: 23-25: Isoforms of PD1 according to the entry of Q15116 in UniProt (https://www.uniprot.org/uniprotkb/Q15116/entry #names_and_taxonomy (11.08.2022). the canonical isoform of PD1 is given in SEQ ID NO: 23.

TABLE 1
Amino Acid sequences of the biomarkers of the application.

| SEQ ID | | |
|---|---|---|
| NO | Description | Sequence |
| 1 | Primary accession number: | MGVEGCTKCIKYLLFVFNFVFWLA |
| | P60033 | GGVILGVALWLRHDPQTTNLLYLE |
| | Length: 236 | LGDKPAPNTFYVGIYILIAVGAVM |
| | Organism: Homo sapiens | MFVGFLGCYGAIQESQCLLGTFFT |
| | Gene: CD81 | CLVILFACEVAAGIWGFVNKDQIA |
| | Protein name: CD81 antigen | KDVKQFYDQALQQAVVDDDANNAK |
| | | AVVKTFHETLDCCGSSTLTALTTS |
| | | VLKNNLCPSGSNIISNLFKEDCHQ |
| | | KIDDLFSGKLYLIGIAAIVVAVIM |
| | | IFEMILSMVLCCGIRNSSVY |
| 2 | Primary accession number: | MWCGWSFSWQPVHWVRSACGCSLT |
| | A6NMH8 | WVSWSLASSSSRSLLKVGELGMAP |
| | Length: 274 | FVHARAGKTKAQLAGGVILGVALW |
| | Organism: Homo sapiens | LRHDPQTTNLLYLELGDKPAPNTF |
| | Gene: CD81 | YVGIYILIAVGAVMMFVGFLGCYG |
| | Protein name: Tetraspanin | AIQESQCLLGTFFTCLVILFACEV |
| | | AAGIWGFVNKDQIAKDVKQFYDQA |
| | | LQQAVVDDDANNAKAVVKTFHETL |
| | | DCCGSSTLTALTTSVLKNNLCPSG |
| | | SNIISNLFKEDCHQKIDDLFSGKL |
| | | YLIGIAAIVVAVIMIFEMILSMVL |
| | | CCGIRNSSVY |
| 3 | Primary accession number: | MAPFVHARAGKTKAQLAGGVILGV |
| | E9PRJ8 | ALWLRHDPQTTNLLYLELGDKPAP |
| | Length: 209 | NTFYVGIYILIAVGAVMMFVGFLG |
| | Organism: Homo sapiens | CYGAIQESQCLLGTFFTCLVILFA |
| | Gene: CD81 | CEVAAGIWGFVNKDQIAKDVKQFY |
| | Protein name: Tetraspanin | DQALQQAVVDDDANNAKAVVKTFH |
| | | ETLDCCGSSTLTALTTSVLKNNLC |
| | | PSGSNIISNLFKEDCHQKIDDLFS |
| | | GKLYLIGIAAIVVAVIM |
| 4 | Primary accession number: E9PIF1 | MEEVTEAQKTHQARLGCEVPCCSS |
| | Length: 242 | LAVSNPTSSQLGGPWWVRHPGPSG |
| | Organism: Homo sapiens | VLGCGECVGTHLVSLSPQGIYILI |
| | Gene: CD81 | AVGAVMMFVGFLGCYGAIQESQCL |
| | Protein name: Tetraspanin | LGTFFTCLVILFACEVAAGIWGFV |
| | | NKDQIAKDVKQFYDQALQQAVVDD |
| | | DANNAKAVVKTFHETLDCCGSSTL |
| | | TALTTSVLKNNLCPSGSNIISNLF |
| | | KEDCHQKIDDLFSGKLYLIGIAAI |
| | | VVAVIMIFEMILSMVLCCGIRNSS |
| | | VY |
| 5 | Primary accession number: | QTQDVPLPAPEAPSPDELAGGVIL |
| | HOYEE2 | GVALWLRHDPQTTNLLYLELGDKP |
| | Length: 148 | APNTFYVGIYILIAVGAVMMFVGF |
| | Organism: Homo sapiens | LGCYGAIQESQCLLGTFFTCLVIL |
| | Protein name: Tetraspanin | FACEVAAGIWGFVNKDQIAKDVKQ |
| | | FYDQALQQAVVDDDANNAKAVVK |
| | | TFHET |
| 6 | Primary accession number: | GGSRWQRGFPQLAGGVILGVALWL |
| | HOYDJ9 | RHDPQTTNLLYLELGDKPAPNTFY |
| | Length: 225 | VGIYILIAVGAVMMFVGFLGCYGA |
| | Organism: Homo sapiens | IQESQCLLGTFFTCLVILFACEVA |
| | Gene: CD81 | AGIWGFVNKDQIAKDVKQFYDQAL |
| | Protein name: Tetraspanin | QQAVVDDDANNAKAVVKTFHETLD |
| | | CCGSSTLTALTTSVLKNNLCPSGS |
| | | NIISNLFKEDCHQKIDDLFSGKLY |
| | | LIGIAAIVVAVIMIFEMILSMVLC |
| | | CGIRNSSVY |
| 7 | Primary accession number: | XQRGFPQLAGGVILGVALWLRHDP |
| | HOYDL9 | QTTNLLYLELGDKPAPNTFYVGIY |
| | Length: 221 | ILIAVGAVMMFVGFLGCYGAIQES |
| | Organism: Homo sapiens | QCLLGTFFTCLVILFACEVAAGIW |
| | Gene: CD81 | GFVNKDQIAKDVKQFYDQALQQAV |
| | Protein name: Tetraspanin | VDDDANNAKAVVKTFHETLDCCGS |
| | | STLTALTTSVLKNNLCPSGSNIIS |
| | | NLFKEDCHQKIDDLFSGKLYLIGI |
| | | AAIVVAVIMVSGRGRRACSLGCPF |
| | | RGALC |
| 8 | Primary accession number: | MCKPATDPRFTRSPLAGGVILGVA |
| | E9PQV4 | LWLRHDPQTTNLLYLELGDKPAPN |
| | Length: 99 | TFYVGIYILIAVGAVMMFVGFLGC |
| | Organism: Homo sapiens | YGAIQESQCLLGTFFTCLVILFAC |
| | Gene: CD81 | EVA |
| | Protein name: CD81 antigen | |
| 9 | Primary accession number: | MMFVGFLGCYGAIQESQCLLGTFF |
| | E9PM31 | TCLVILFACEVAAGIWGFVNKDQI |
| | Length: 92 | AKDVKQFYDQALQQAVVDDDAN |
| | Organism: Homo sapiens | NAKAVVKTFHETLDCCGSSTLT |
| | Gene: CD81 | |
| | Protein name: CD81 antigen | |
| 10 | Primary accession number: | MMFVGFLGCYGAIQESQCLLGTFF |
| | E9PPF5 | TCLVILFACEVAAGIWGFVNKDQI |
| | Length: 51 | AKD |
| | Organism: Homo sapiens | |
| | Gene: CD81 | |
| | Protein name: CD81 antigen | |
| 11 | Primary accession number: | MMFVGFLGCYGAIQESQCLLGTFF |
| | E9PJK1 | TCLVILFACEVAAGIWGFVNKDQI |
| | Length: 165 | AKDVKQFYDQALQQAVVDDDANNA |
| | Organism: Homo sapiens | KAVVKTFHETLDCCGSSTLTALTT |
| | Gene: CD81 | SVLKNNLCPSGSNIISNLFKEDCH |
| | Protein name: Tetraspanin | QKIDDLFSGKLYLIGIAAIVVAVI |
| | | MIFEMILSMVLCCGIRNSSVY |
| 12 | Primary accession number: P16410 | MACLGFQRHKAQLNLATRTWPCTL |
| | Length: 223 | LFFLLFIPVFCKAMHVAQPAVVLA |
| | Organism: Homo sapiens | SSRGIASFVCEYASPGKATEVRVT |
| | Gene: CTLA4 | VLRQADSQVTEVCAATYMMGNELT |
| | Protein name: Cytotoxic T- | FLDDSICTGTSSGNQVNLTIQGLR |
| | lymphocyte protein 4 | AMDTGLYICKVELMYPPPYYLGIG |
| | Canonical Sequence | NGTQIYVIDPEPCPDSDFLLWILA |
| | | AVSSGLFFYSFLLTAVSLSKMLKK |
| | | RSPLTTGVYVKMPPTEPECEKQFQ |
| | | PYFIPIN |
| 13 | Primary accession number: P16410 | MACLGFQRHKAQLNLATRTWPCTL |
| | Length:56 | LFFLLFIPVFCKAPPTEPECEKQF |
| | Organism: Homo sapiens | QPYFIPIN |
| | Gene: CTLA4 | |
| | Protein name: Cytotoxic T- | |
| | lymphocyte protein 4 | |
| | Also known as: ss-CTLA-4 | |
| | The sequence of this isoform differs | |
| | from the canonical sequence as | |
| | follows: | |
| | 38-204: Missing. | |
| 14 | Primary accession number: P16410 | MACLGFQRHKAQLNLATRTWPCT |
| | Length: 58 | LLFFLLFIPVFCKAKEKKPSYNR |
| | Organism: Homo sapiens | GLCENAPNRARM |
| | Gene: CTLA4 | |
| | Protein name: Cytotoxic T- | |
| | lymphocyte protein 4 | |
| | The sequence of this isoform differs | |
| | from the canonical sequence as | |
| | follows: | |
| | 38-204: Missing. | |
| | 205-223: | |
| | PPTEPECEKQFQPYFIPIN → KEK | |
| | KPSYNRGLCENAPNRARM | |
| 15 | Primary accession number: P16410 | MACLGFQRHKAQLNLATRTWPCTL |
| | Length: 79 | LFFLLFIPVFCKAMHVAQPAVVLA |
| | Organism: Homo sapiens | SSRGIASFVSKEKKPSYNRGLCEN |
| | Gene: CTLA4 | APNRARM |
| | Protein name: Cytotoxic T- | |
| | lymphocyte protein 4 | |
| | The sequence of this isoform differs | |
| | from the canonical sequence as | |
| | follows: | |
| | 58-58: C → S | |
| | 59-204: Missing. | |
| | 205-223: | |
| | PPTEPECEKQFQPYFIPIN → KEK | |
| | KPSYNRGLCENAPNRARM | |
| 16 | Primary accession number: P16410 | MACLGFQRHKAQLNLATRTWPCT |
| | Length: 174 | LLFFLLFIPVFCKAMHVAQPAVV |
| | Organism: Homo sapiens | LASSRGIASFVCEYASPGKATEV |
| | Gene: CTLA4 | RVTVLRQADSQVTEVCAATYMMG |
| | Protein name: Cytotoxic | NELTFLDDSICTGTSSGNQVNLT |
| | T-lymphocyte protein 4 | IQGLRAMDTGLYICKVELMYPPP |
| | The sequence of this isoform differs | YYLGIGNGTQIYVIAKEKKPSYN |
| | from the canonical sequence as | RGLCENAPNRARM |
| | follows: | |
| | 153-174: | |
| | DPEPCPDSDFLLWILAAVSSGL → | |
| | AKEKKPSYNRGLCENAPNRARM | |
| | 175-223: Missing. | |
| 17 | Primary accession number: | MRIFAVFIFMTYWHLLNAFTVTVP |
| | Q9NZQ7 | KDLYVVEYGSNMTIECKFPVEKQL |
| | Length: 290 | DLAALIVYWEMEDKNIIQFVHGEE |
| | Organism: Homo sapiens | DLKVQHSSYRQRARLLKDQLSLGN |
| | Gene: CD274 (B7H1, PDCD1L1, | AALQITDVKLQDAGVYRCMISYGG |
| | PDCD1LG1, PDL1) | ADYKRITVKVNAPYNKINQRILVV |
| | Protein name: Programmed cell | DPVTSEHELTCQAEGYPKAEVIWT |
| | death 1 ligand 1 | SSDHQVLSGKTTTTNSKREEKLFN |
| | Canonical sequence | VTSTLRINTTTNEIFYCTFRRLDP |
| | | EENHTAELVIPELPLAHPPNERTH |
| | | LVILGAILLCLGVALTFIFRLRKG |
| | | RMMDVKKCGIQDTNSKKQSDTHLE |
| | | ET |
| 18 | Primary accession number: | MRIFAVFIFMTYWHLLNAPYNKIN |
| | Q9NZQ7 | QRILVVDPVTSEHELTCQAEGYPK |
| | Length: 176 | AEVIWTSSDHQVLSGKTTTTNSKR |
| | Organism: Homo sapiens | EEKLFNVTSTLRINTTTNEIFYCT |
| | Gene: CD274 (B7H1, PDCD1L1, | FRRLDPEENHTAELVIPELPLAHP |
| | PDCD1LG1, PDL1) | PNERTHLVILGAILLCLGVALTFI |
| | Protein name: Programmed cell | FRLRKGRMMDVKKCGIQDTNSKKQ |
| | death 1 ligand 1 | SDTHLEET |
| | The sequence of this isoform differs | |
| | from the canonical sequence as | |
| | follows: | |
| | 19-132: Missing | |
| 19 | Primary accession number: | MRIFAVFIFMTYWHLLNAFTVTVP |
| | Q9NZQ7 | KDLYVVEYGSNMTIECKFPVEKQL |
| | Length: 178 | DLAALIVYWEMEDKNIIQFVHGEE |
| | Organism: Homo sapiens | DLKVQHSSYRQRARLLKDQLSLG |
| | Gene: CD274 (B7H1, PDCD1L1, | NAALQITDVKLQDAGVYRCMISYG |
| | PDCD1LG1, PDL1) | GADYKRITVKVNAPYNKINQRILV |
| | Protein name: Programmed cell | VDPVTSEHELTCQAEGYPKAEVIW |
| | death 1 ligand 1 | TSSDHQVLSGD |
| | The sequence of this isoform differs | |
| | from the canonical sequence as | |
| | follows: | |
| | 178-178: K → D | |
| | 179-290: Missing | |
| 20 | Primary accession number: | MIFLLLMLSLELQLHQIAALFTVTV |
| | Q9BQ51 | PKELYIIEHGSNVTLECNFDTGSH |
| | Length: 273 | VNLGAITASLQKVENDTSPHRERA |
| | Organism: Homo sapiens | TLLEEQLPLGKASFHIPQVQVRDE |
| | Gene: PDCD1LG2 (B7DC, CD273, | GQYQCIIIYGVAWDYKYLTLKVKA |
| | PDCD1L2, PDL2) | SYRKINTHILKVPETDEVELTCQA |
| | Protein name: Programmed cell | TGYPLAEVSWPNVSVPANTSHSRT |
| | death 1 ligand 2 | EPGLYQVTSVLRLKPPPGRNFSCV |
| | Canonical Isoform | FWNTHVRELTLASIDLQSQMEPRT |
| | | HPTWLLHIFIPFCIIAFIFIATVI |
| | | ALRKQLCQKLYSSKDTTKRPVTTT |
| | | KREVNSAI |
| 21 | Primary accession number: | MIFLLLMLSLELQLHQIAALFTVTV |
| | Q9BQ51 | PKELYIIEHGSNVTLECNFDTGSH |
| | Length: 183 | VNLGAITASLQKVENDTSPHRERA |
| | Organism: Homo sapiens | TLLEEQLPLGKASFHIPQVQVRDE |
| | Gene: PDCD1LG2 (B7DC, CD273, | GQYQCIIIYGVAWDYKYLTLKVKG |
| | PDCD1L2, PDL2) | QMEPRTHPTWLLHIFIPFCIIAFI |
| | Protein name: Programmed cell | FIATVIALRKQLCQKLYSSKDTTK |
| | death 1 ligand 2 | RPVTTTKREVNSAI |
| | The sequence of this isoform differs | |
| | from the canonical sequence as | |
| | follows: | |
| | 121-211: | |
| | ASYRKINTHILKVPETDEVELTCQ | |
| | ATGYPLAEVSWPNVSVPANTSHS | |
| | RTPEGLYQVTSVLRLKPPPGRNFS | |
| | CVFWNTHVRELTLASIDLQS → G | |
| 22 | Primary accession number: | MIFLLLMLSLELQLHQIAALFTVTV |
| | Q9BQ51 | PKELYIIEHGSNVTLECNFDTGSH |
| | Length: 182 | VNLGAITASLQKVENDTSPHRERA |
| | Organism: Homo sapiens | TLLEEQLPLGKASFHIPQVQVRDE |
| | Gene: PDCD1LG2 (B7DC, CD273, | GQYQCIIIYGVAWDYKYLTLKVKD |
| | PDCD1L2, PDL2) | GTQDPSNLAASHFHPLLHHCFHFH |
| | Protein name: Programmed cell | SHSDSPKKTTLSKAVFFKRHNKK |
| | death 1 ligand 2 | TCHHNKEGSEQCYL |
| | The sequence of this isoform differs | |
| | from the canonical sequence as | |
| | follows: | |
| | 121-182: | |
| | ASYRKINTHILKVPETDEVELTCQ | |
| | ATGYPLAEVSWPNVSVPANTSHS | |
| | RTPEGLYQVTSVLRL → | |
| | DGTQDPSNLAASHFHPLLHHCFH | |
| | FHSHSDSPKKTTLSKAVFFKRHN | |
| | KKTCHHNKEGSEQCYL | |
| | 183-273: Missing | |
| 23 | Primary accession number: Q15116 | MQIPQAPWPVVWAVLQLGWRPG |
| | Length: 288 | WFLDSPDRPWNPPTFSPALLVVTE |
| | Organism: Homo sapiens | GDNATFTCSFSNTSESFVLNWYRM |
| | Gene: PDCD1 (PD1) | SPSNQTDKLAAFPEDRSQPGQDCR |
| | Protein name: Programmed cell | FRVTQLPNGRDFHMSVVRARRNDS |
| | death protein 1 | GTYLCGAISLAPKAQIKESLRAEL |
| | Canonical Isoform | RVTERRAEVPTAHPSPSPRPAGQF |
| | | QTLVVGVVGGLLGSLVLLVWVLAV |
| | | ICSRAARGTIGARRTGQPLKEDPS |
| | | AVPVFSVDYGELDFQWREKTPEPP |
| | | VPCVPEQTEYATIVFPSGMGTSSP |
| | | ARRGSADGPRSAQPLRPEDGHCSW |
| | | PL |
| 24 | Primary accession number: | MQIPQAPWPVVWAVLQLGWRPGW |
| | E7ER21 | FLDSPDRPWNPPTFSPALLVVTE |
| | Length: 66 | GDNATFTCSFSNTSESFVLN |
| | Organism: Homo sapiens | |
| | Gene: PDCD1 | |
| | Protein name: Programmed cell | |
| | death protein 1 | |
| 25 | Primary accession number: | XNTSESFVLNWYRMSPSNQTDKL |
| | HOY2W6 | AAFPEDRSQPGQDCRFRVTQLPN |
| | Length: 122 | GRDFHMSVVRARRNDSGTYLCGA |
| | Organism: Homo sapiens | ISLAPKAQIKESLRAELRVTGTI |
| | Gene: PDCD1 | GARRTGQPLKEDPSAVPVFSVDY |
| | Protein name: Programmed cell | GELDFQW |
| | death protein 1 | |

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

### Materials and Methods

MSC growth and expansion: Human BM aspirates from healthy donors were obtained following informed consent according to the Declaration of Helsinki. Their usage was approved by the ethics committee of the University of Duisburg-Essen (12-5295-BO). To raise MSCs, aliquots of obtained BM aspirates were seeded into cell culture flasks containing endothelial basal media (EBM-2, Lonza, Cologne, Germany) supplemented with 10% human platelet lysate (PL; produced in house) and the provided bullet kit (includes human endothelial growth factor ([EGF], hydrocortisone, gentamicin, amphotericin-b [GA-1000], vascular endothelial growth factor [VEGF], human fibroblast growth factor [hFGF], insulin-like growth factor [R3-IGF-1], ascorbic acid and heparin). After incubation for 24 hours at 37° C. in a 5% CO₂ atmosphere, non-adherent cells were removed by medium exchange to DMEM low glucose (PAN Biotech), supplemented with 10% PL, 100 U/mL penicillin-streptomycin-glutamine (Thermo Fisher Scientific, Darmstadt, Germany) and 5 IU/mL Heparin (Ratiopharm, Ulm, Germany). Cultures were continuously cultured at 37° C. in a 5% CO₂ atmosphere and regularly screened microscopically until the first MSC colonies became visible. Following trypsin/EDTA (Sigma-Aldrich, Taufkirchen, Germany) treatment including a washing step, adherent cells were re-seeded at densities of approximately 1000 cells/cm into 4-layer stack cell Factory™ systems (Thermo Fischer Scientific). Within the second passage, MSCs were analysed according to the criteria of the International Society of Cell and Gene Therapy (ISCT) 11. MSCs were fluorescently labelled with anti-CD14, anti-CD31, anti-CD34, anti-CD44, anti-CD45, anti-CD73, anti-CD90, anti-CD105 and anti-HLA-ABC antibodies (Table 2) and analysed by flow cytometry (Cytoflex; Software Cytexpert 2.3, Beckman-Coulter, Krefeld, Germany).

TABLE 2
List of dye-conjugated antibodies

| Antigen | Conjugate | Host/isotype | Clone | Supplier |
|---|---|---|---|---|
| HUMAN | Pacific Orange | Mouse IgG1 | MEM-15 | Exbio |
| CD14 | | | | |
| HUMAN | Phycoerythrin | Mouse IgG1 | 1F11 | Beckman |
| CD31 | | | | Coulter |
| HUMAN | Allophycocyanin | Mouse IgG1 | 581 | Beckman |
| CD34 | 750 | | | Coulter |
| HUMAN | Allophycocyanin | Mouse IgG2b, | G44-26 | BD |
| CD44 | | kappa | | Biosciences |
| HUMAN | Brilliant Violet | Mouse IgG1, | HI30 | BioLegend |
| CD45 | 785 | kappa | | |
| HUMAN | Fluorescein | Mouse IgG1, | AD2 | BD |
| CD73 | isothiocyanate | kappa | | Biosciences |
| HUMAN | Brilliant Violet | Mouse IgG1, | 5E10 | BioLegend |
| CD90 | 605 | kappa | | |
| HUMAN | Brilliant Violet | Mouse IgG1, | 43A3 | Biolegend |
| CD105 | 421 | kappa | | |

Upon passage 3, the MSCs' osteogenic and adipogenic differentiation potentials were confirmed by conventional MSC differentiation assays according to [12, 13]. Upon reaching densities of approximately 50% confluency, media were changed every 48 hours. At 80% confluency, MSCs were passaged. For the preservation of the change medium (CM), cells and larger debris were removed by 2000×g centrifugation of cell suspensions for 15 minutes (Rotor: JS-5.3; Beckman Coulter). MSC-free CM were stored at −20° C. until usage. CMs were screened regularly for mycoplasma contamination (Venor®GeM OneStep, Minerva Biolabs, Berlin, Germany).

Preparation of EVs: For EV harvesting CMs were thawed and further purified following 45 min 6,800×g centrifugation (Rotor: JS-5.3) by a subsequent 0.22 μm filtration step using rapid flow filter (Nalgene, Thermo Fisher Scientific). EVs were precipitated in 10% polyethylene glycol 6000 (PEG) and 75 mM sodium chloride (NaCl) by overnight incubation and subsequent centrifugation at 1,500×g and 4° C. for 30 min according to the method of [13, 14]. Pelleted EVs were re-suspended and washed with sterile 0.9% NaCl solution (Braun, Nelsungen, Germany) to remove contaminating soluble proteins. Next, EVs were re-precipitated by ultracentrifugation at 110,000×g for 130 min (XPN-80, Ti45 rotor, k-factor: 133). Finally, EV pellets were re-suspended in 10 mM HEPES 0.9% NaCl buffer (Thermo Fisher Scientific). Concentration was adjusted so that 1 mL final sample contained the EV yield prepared from CM of approximately 4×10⁷ MSC equivalents. MSC-EV preparations were stored at −80° C. Repetitive thawing and freezing cycles were avoided. For control purposes, fresh PL supplemented media were processed in parallel (including incubation for 48 hours at 37° C., 5% CO₂, saturated water vapour atmosphere).

Multi-donor mixed lymphocyte reaction (mdMLR): The immunomodulatory potential of Exoria labelled and non-labelled MSC-EV preparations were compared in a multi-donor mixed lymphocyte reaction assay (MLR) according to [15]. Briefly, Ficoll prepared peripheral blood mononuclear cells (PBMC) of 12 healthy donors were mixed in equal proportions, aliquoted and stored in the vapour phase of liquid nitrogen until usage. After thawing 600,000 cells were seeded per well of a 96-well U-bottom shape plates (Corning, Kaiserslautern, Germany) and cultured in 10% human AB serum (produced in house) and 100 U/mL penicillin and 100 μg/mL streptomycin (Thermo Fisher Scientific) supplemented RPMI 1640 medium (Thermo Fisher Scientific) in a final volume of 200 μL per well, either in the presence or absence of MSC-EV preparations to be tested. After 5 days, cells were harvested, stained with a collection of different fluorescent labelled antibodies (CD4-BV785; BioLegend, San Diego, CA, USA; CD25-PE-Cy5.5; BD Bioscience; and CD54-AF700; EXBIO) and analysed on a Cytoflex flow cytometer (Software CytExpert 2.3, Beckman-Coulter). Activated and non-activated CD4⁺ T cells were discriminated by means of their CD25 and CD54 expression, respectively. Typically, 5 μL of MSC-EV preparations to be tested were applied into respective wells.

Example 4: Imaging Flow Cytometry (IFCM): IFCM was performed on a AMNIS ImageStreamX Mark II Flow Cytometer (AMNIS/Luminex, Seattle, WA, USA) according to [16, 17]. Anti-CD152-PE (Clone BNI3, BD Biosciences, Heidelberg, Germany) was used to characterize the MSC-EV preparations. Generally, 5 μL of the MSC-EV preparation (0.005 units) were used and incubated for 2 hours at room temperature with 12 nM of the antibody. According to the recommendations of the MIFlowCyt-EV guidelines [18], unstained EV samples, NaCl-HEPES buffer with antibodies but without EV sample as well as stained sample supplemented with 1% NP40 (Calbiochem, San Diego, CA, USA) were analyzed as controls. After staining, samples were diluted with PBS to 200 μL and analyzed using the built-in autosampler for 96-well round bottom plates. Acquisition time was selected as 5 minutes per well. Data were acquired at 60× magnification, low flow rate and with removed beads option deactivated.

Data were analyzed using the IDEAS software (version 6.2) according to [15, 16]. Fluorescent events were plotted against the side scatter (SSC). A combined mask feature was used (MC and NMC) to improve the detection of fluorescent images. Images were analyzed for coincidences (swarm detection) by using the spot counting feature. Every data point with multiple objects was excluded from the analyses. Average concentrations were calculated according to the acquisition volume and time.

Western Blotting: Western blot analyses were performed according to by solubilising samples with Laemmli sample buffer containing DTT (AppliChem, Darmstadt, Germany) and separated on an 1D sodium dodecyl sulfate polyacrylamide gel electrophoresis (SDS-PAGE) gel before transfer to polyvinylidene fluoride membrane (PVDF; Millipore, Darmstadt, Germany). Page Ruler, Prestained Protein ladder (Thermo Fisher Scientific) was used as a marker. PVDF membranes were blocked in PBS-0.1% Tween-20 (PBS-T) or TBS-0.1% Tween-20 (TBS-T) containing 5% (w/v) skim milk powder (Sigma-Aldrich). To rinse off residual blocking solution membranes were washed three times (5, 10 and 20 min) in PBS-T/TBS-T. For the detection anti-CTLA-4 (Abcam, Cambridge, U.K) was used. To rinse off residual primary antibodies, membranes were washed three times (5, 10 and 20 min) in PBS-T/TBS-T.

Data Analysis and Statistics: Computational data plotting, analysis and visualization was performed using Graph Pad Prism (version 8.4.0).

### The Examples Show:

### Example 1: Immunomodulatory Active MSC-EV Preparations Show Increased Concentration of CTLA-4-Positive Objects as Measured by IFC

MSCs were isolated from different donors and cultured for at least three passages. MSC-EVs from 32 independent human platelet lysate cultured MSCs were prepared and characterized for CTLA-4. In addition, MSC-EVs were repeatedly isolated from the same donor to investigate intradonor heterogeneity. After isolation by polyethylene glycol precipitation followed by ultracentrifugation, the resulting pellets were resuspended by unit definition in NaCl/HEPES buffer (equivalent of 4×10⁷ cells per mL). The immunomodulatory effect on CD4⁺ T cell activity and proliferation was determined by multi-donor mixed lymphocyte reaction (mdMLR), classifying the preparations into immunomodulatory and non-immunomodulatory. Each of the classified MSC-EV preparation was measured for the concentration of CTLA-4 in a technical duplicate by using imaging flow cytometry (IFC).

All preparation results were combined, both from different donors and from different preparations from the same donor. The concentration of CTLA-4 in the immunomodulating MSC-EV preparations could be determined as 6.86·10⁷±6.33·10⁷ positive CTLA-4 objects, which is highly significant increased (p<0.0001, two-tailed Mann-Whitney test) compared to concentration of non-immunomodulating preparations the (4.29×10⁶±7.31×10⁵ objects/mL) (FIG. 1). According to these data, measurement of CTLA-4 by IFC can distinguish immunomodulating preparations from non-immunomodulating ones.

### Example 2: Western Blot Shows CTLA-4 in Cell Culture Supernatants, but Only Recovered in Immunomodulating MSC-EV Preparations

Western blots of the unprocessed supernatants and the corresponding MSC-EV preparations were performed to further qualify the preparations. Again, anti-CTLA-4 was used for characterization. All samples were applied with an amount of 25 μg of protein as determined by BCA assay. FIG. 2 shows an exemplary Western blot. For the unprocessed supernatants as well as the corresponding MSC-EV preparation with the immunomodulatory effect, a band at approx. 36 kDa can be detected. The Western blot shows that even in non-immunomodulating preparations, CTLA-4 can originally be detected in the supernatants, but these are no longer detectable after purification via PEG precipitation followed by ultracentrifugation. Since Western blot also detects non-EV-associated proteins, the CTLA-4 in the supernatant could be the soluble form that is absent or reduced after purification. This could then also confirm the results of the IFC analyses, where only the EV-bound form can be detected.

### Example 3: Overexpression of CTLA-4 in MSCs Enhances the Immunomodulatory Capacity of EV Preparations Compared to Wild Type

To further investigate the effect and correlation of CTLA-4 in MSC-EV preparations, immortalized cell lines were created that overexpress CTLA-4 via lentiviral transduction. The wild type itself is an immortalized MSC cell line grown from single cell depots that shows an immunomodulatory effect in mdMLR, but its strength varies depending on the MSC-EV preparation. To exclude possible intradonor heterogeneity, cell lines were obtained from single cell deposits after inducing the overexpression of CTLA-4. MSC-EV preparations were generated, purified, and characterized as described before. Here, quantification of EVs by IFC shows an increased concentration of CTLA-4 in EVs from CTLA-4 overexpressing cells (5.03×10⁸±3.14×10⁷ objects/mL) compared to WT (3.07×10⁷±3.40×10⁶ objects/mL), even surpassing the preparations from primary culture (FIG. 3, panel A). To detect a possible stronger immunomodulatory effect compared to the wild type, an mdMLR was performed. For this purpose, mdMLRs were prepared from three independent MSC-EV preparations from the WT and the CTLA-4 overexpressing cell line, and the effect on CD4+ T cells and CD4+CD25+CD54+ cells was examined. The untreated PBMCs of the mdMLRs were used as reference to calculate the fold change of present cells (FIG. 3, panel B). Overall, fewer T cells proliferated when treated with the preparations of CTLA-4 overexpressing cell line compared with WT. WT showed little change in CD4+ T cells compared with the untreated control (0.893±0.084). The preparations of CTLA-4 overexpressing cells reduce the amount of CD4+ T cells to 0.633±0.071. A more pronounced effect is seen in activated CD4+ T cells (CD25+CD54+), which were reduced to 0.183±0.123 compared with WT, where the population was reduced to only 0.703±0.265. Overall, overexpression of CTLA-4 in MSCs leads to a higher immunomodulatory effect in the MSC-EV preparations.

### Example 4: Overexpression of CTLA-4, PD-L1 and PD-L2 Lead to Different Immunomodulatory Activities of the Respective MSC-EV Preparations

To investigate the effect of different immune checkpoint inhibitors in MSC-EV preparations, immortalized cell lines overexpressing CTLA-4, PD-L1, or PD-L2 were generated by lentiviral transduction. Transduced MSCs were analyzed according to International Society of Cell and Gene Therapy (ISCT) criteria [11]. In addition, the expression of CTLA-4, PD-L1, or PD-L2 was verified using a fluorescent labeled antibody against the respective surface protein (FIG. 4A). PD-L1 and PD-L2 have different expression levels on MSCs (FIG. 4B) which results in a dim and a high population. No distinction was made for further analyses of EV preparations.

The EVs were isolated as described before and characterized by using IFCM. The preparations show positive objects for the corresponding protein (FIG. 5A). For the immunomodulatory capability, the MSC-EVs were tested in the mdMLR and the effect on activated CD4+ and CD8+ T cells (CD25+CD54+) was examined. The untreated PBMCs of the mdMLRs were used as reference to calculate the change of present cells (FIG. 5B). Overall, fewer T cells proliferated when treated with the preparations of CTLA-4 or PD-L1 overexpressing cell line compared with WT. The preparations of CTLA-4 overexpressing cells reduce the amount of activated CD4+ T cells (CD25+CD54+) to 0.183±0.123 compared to the WT, where the population was reduced to only 0.703±0.265. For preparations of PD-L1 cells, the activity of CD4+CD25+CD54+ cells were reduced to 0.421 compared to the WT (1.22). Activated CD8 T cells show a similar pattern. CTLA-4 reduces the amount of activated CD8+ T cells to 0.173±0.029 (WT: 0.797±0.005), while PD-L1 decreases it to 0.284 (WT: 0.932).

### REFERENCES

¹Rybak, K, et al., Functions of Extracellular Vesicles in Immunity and Virulence. Plant Physiol. 2019; 179, 1236. doi: 10.1104/pp. 18.01557
²Yáñez-Mó et al., Biological properties of extracellular vesicles and their physiological functions. JEV. 2015, 4:1. doi: 10.3402/jev.v4.27066
³Fais et al., Evidence-Based Clinical Use of Nanoscale Extracellular Vesicles in Nanomedicine. ACS nano. 2016, 10, 4, 3886-3899. doi: 10.1021/acsnano.5bo8015
⁴Srivastava, A, et al., Progress in extracellular vesicle biology and their application in cancer medicine. WIRES Nanomed Nanobiotechnol. 2020; 12: e1621. doi: 10.1002/wnan.1621
⁵Brennan, K, et al., A comparison of methods for the isolation and separation of extracellular vesicles from protein and lipid particles in human serum. Sci Rep. 2020; 10, 1039. doi: 10.1038/s41598-020-57497-7
⁶Rankin-Turner et al., A call for the standardised reporting of factors affecting the exogenous loading of extracellular vesicles with therapeutic cargos. 2021, 173, 479-491. doi: 10.1016/j.addr.2021.04.012
⁷Lener et al., Applying extracellular vesicles based therapeutics in clinical trials—an ISEV position paper. J Extracell Vesicles. 2015, 4:30087. doi: 10.3402/jev.v4.30087.
⁸Wiklander O, et al., Advances in therapeutic applications of extracellular vesicles, Science Translational Medicine. 2019; 11, eaav8521. doi: 10.1126/scitranslmed.aav8521
⁹Park K S, et al. Enhancement of therapeutic potential of mesenchymal stem cell-derived extracellular vesicles, Stem Cell Res ther. 2018; 10, 288. doi: 10.1186/s13287-019-1398-3
¹⁰EP2687219A1
¹¹Dominici, M., et al., Minimal criteria for defining multipotent mesenchymal stromal cells. The International Society for Cellular Therapy position statement, Cytotherapy. 2006. 8(4): p. 315-7. doi: 10.1080/14653240600855905
¹²Radtke, S., et al., Human mesenchymal and murine stromal cells support human lympho-myeloid progenitor expansion but not maintenance of multipotent haematopoietic stem and progenitor cells. Cell Cycle, 2016. 15 (4): p. 540-5. doi: 10.1080/15384101.2015.1128591
¹³Kordelas, L., et al., MSC-derived exosomes: a novel tool to treat therapy-refractory graft-versus-host disease. Leukemia. 2014. 28 (4): p. 970-3. doi: 10.1038/leu.2014.41
¹⁴Ludwig, A. K., et al., Precipitation with polyethylene glycol followed by washing and pelleting by ultracentrifugation enriches extracellular vesicles from tissue culture supernatants in small and large scales. J Extracell Vesicles. 2018. 7(1): p. 1528109. doi: 10.1080/20013078.2018.1528109
¹⁵Madel, R. J., et al., Independent human mesenchymal stromal cell-derived extracellular vesicle preparations differentially affect symptoms in an advanced murine Graft-versus-Host-Disease model. bioRxiv. 2020: p. 2020.12.21.423658. doi: 10.1101/2020.12.21.423658
¹⁶Tertel, T., A. Görgens, and B. Giebel, Chapter Four—Analysis of individual extracellular vesicles by imaging flow cytometry, Methods in Enzymology. 2020, Academic Press. p. 55-78. doi: 10.1016/bs.mie.2020.05.013
¹⁷Tertel, T., et al., High-Resolution Imaging Flow Cytometry Reveals Impact of Incubation Temperature on Labeling of Extracellular Vesicles with Antibodies. Cytometry A. 2020. 97(6): p. 602-609. doi: 10.1002/cyto.a.24034. Epub 2020 May 16.
¹⁸Welsh, J. A., et al., MIFlowCyt-EV: a framework for standardized reporting of extracellular vesicle flow cytometry experiments. J Extracell Vesicles. 2020. 9(1): p. 1713526. doi: 10.1080/20013078.2020.1713526

1. A method for assessing the therapeutic efficacy of an Extracellular Vesicle (EV) or an EV-preparation, comprising the following steps:
(i) Providing the EV or EV-preparation;
(ii) Detecting, optionally by using a binding agent, the presence or absence of at least one EV surface marker, preferably wherein the at least one EV surface marker is selected from the group of CTLA-4, PD1, PD-L1 and PD-L2, wherein the EV surface marker is associated with the EV or EV-preparation;
Wherein the presence of the at least one EV surface marker associated with the EV or EV-preparation is indicative of a therapeutically sufficient efficacy of the EV or EV-preparation.

2. The method of claim 1, wherein the EV is characterized by being an EV secreted from a secretory cell that is a mammalian cell, preferably a human cell.

3. The method of claim 2, wherein the secretory cell is a mesenchymal stromal cell (MSC).

4. The method of claim 2, wherein the secretory cell is characterized by an expression, preferably an overexpression of the at least one EV surface marker.

5. The method of claim 2, wherein the secretory cell is characterized by a native expression and/or is capable of a native expression of the at least one EV surface marker and/or wherein the secretory cell is characterized by a recombinant expression of the at least one EV surface marker and/or is capable of a recombinant expression the at least one EV surface marker.

6. The method of claim 2, wherein the at least one EV surface marker is a native protein of the secretory cell.

7. The method of claim 1, wherein detecting the presence or absence of at least one EV surface maker in step ii) comprises detecting a (specific) binding between the binding-agent and the at least one EV surface marker, preferably wherein detecting the (specific) binding of the binding-agent to the at least one EV surface marker is based on an immunological-, spectroscopic-, and/or spectrometric method, preferably wherein the spectroscopic method comprises UV/Vis spectroscopy, fluorescence spectroscopy, nuclear magnetic resonance (NMR) spectroscopy, infrared (IR) spectroscopy, Raman-spectroscopy and/or surface plasmon resonance spectroscopy, preferably the immunological method comprises (enzyme-linked immunosorbent assay) ELISA, enzyme immunoassay (EIA), fluorescence immunoassay (FIA), chemiluminescence immunoassay (CIA), radioimmunoassay (RIA), Western-Blot and/or peptide-array, preferably wherein the spectrometric method is conducted with a variant of matrix-assisted laser desorption/ionization (MALDI) and/or electrospray ionization (ESI).

8. The method of claim 1, wherein the binding-agent is an antigen binding construct, such as a protein or nucleic acid, and preferably is a ligand molecule specific for the at least one EV surface marker or derivative thereof, an antibody or antigen binding fragment or derivative thereof, a T-cell receptor or any antigen binding fragment or derivative thereof, an aptamer, nanobody, or other molecule antigen binding construct specifically binding to the at least one EV surface marker, preferably wherein the binding-agent is an antibody or antigen binding fragment or derivative thereof.

9. The method of claim 1, wherein the presence or absence of the at least one EV surface marker is compared to the presence or absence of the at least one EV surface marker of a control or reference, preferably wherein the reference is any of the following:
(i) an EV reference preparation comprising a predetermined amount of the at least one EV surface marker, optionally wherein the EV reference preparation is not comprising any of the EV surface marker;
(ii) an EV reference preparation comprising a predetermined amount of the at least one EV surface marker that is associated with the binding agent;
(iii) an EV-reference-preparation with a predetermined therapeutic efficacy, optionally wherein the EV-reference is not comprising any therapeutic efficacy;

10. A method for producing an EV or EV-preparation with therapeutically sufficient efficacy, comprising the following steps:
(i) Providing a secretory cell;
(ii) Culturing the secretory cell in a culture medium (and under conditions) that allows for a secretion of EVs into the culture medium;
(iii) Harvesting of the secreted EVs to obtain a harvested EV or EV-preparation, preferably wherein harvesting of the EV or EV-preparation is conducted by ultracentrifugation, size-based fractionation, anion-exchange chromatography and/or PEG-precipitation;
(iv) Performing a method of claim 1 with the harvested EV or EV-preparation to determine its therapeutic efficacy;
(v) Selecting the harvested EV or EV-preparation with therapeutically sufficient efficacy;
Wherein the method, prior to culturing the secretory cell, optionally comprises an additional step of enhancing in the secretory cell the secretion of therapeutically efficient EVs.

11. (canceled)

12. (canceled)

13. (canceled)

14. (canceled)

15. A method for manufacturing an EV or EV-preparation with therapeutically sufficient efficacy, comprising the following steps:
(i) Providing a secretory cell;
(ii) Culturing the secretory cell in a culture medium that allows for the secretion of an EV into the culture medium;
(iii) Sorting the secreted EV into at least two fractions, depending on the presence or absence of an EV surface marker that is associated with the EV;
Wherein a fraction of the at least two fractions is the Extracellular Vesicle (EV) or the EV-preparation with therapeutically sufficient efficacy.

16. The method of claim 15, comprising a step of genetically modifying the secretory cell to express the EV surface marker.

17. The method of claim 15, wherein the sorting comprises performing an affinity chromatography.

18. The method of claim 15, wherein the method comprises an additional step of isolating the EVs that is conducted after step (ii) and/or after step (iii).

19. The method of claim 15, wherein the method comprises an additional step of enhancing in the secretory cell the secretion of therapeutically efficient EVs.

20. The method of claim 15, wherein the secretory cell is characterized by a recombinant expression of the at least one EV surface marker and/or is capable of a recombinant expression the at least one EV surface marker.

21. An EV preparation with therapeutically sufficient efficacy obtainable by a method of claim 10 or 15.

22. (canceled)

23. A method of treatment of a condition or disease in a subject, comprising administering to the subject an EV preparation obtainable by a method of claim 10 or 15.

24. The method of claim 23, wherein the disease is an is an immune mediated, endocrine, orthopaedic, neurodegenerative, cardiovascular and/or respiratory disease or injury and/or wherein the disease is a disease or injury of the liver, bone marrow, lung, spleen, brain, pancreas, stomach and/or intestine.

25. The method of claim 23, wherein the disease is a T cell mediated disease.

26. The method of claim 23, wherein the disease is an allergy, asthma, an autoimmune disease, coeliac disease, glomerulonephritis, hepatitis, inflammatory bowel disease, reperfusion injury and/or Graft-versus-Host disease.


SEQUENCE LISTING (25 sequences)

SEQ ID NO: 1 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 2 - protein, 274 aa, Homo sapiens
MWCGWSFSWQPVHWVRSACGCSLTWVSWSLASSSSRSLLKVGELGMAPFVHARAGKTKAQLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 3 - protein, 209 aa, Homo sapiens
MAPFVHARAGKTKAQLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIM

SEQ ID NO: 4 - protein, 242 aa, Homo sapiens
MEEVTEAQKTHQARLGCEVPCCSSLAVSNPTSSQLGGPWWVRHPGPSGVLGCGECVGTHLVSLSPQGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 5 - protein, 148 aa, Homo sapiens
QTQDVPLPAPEAPSPDELAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHET

SEQ ID NO: 6 - protein, 225 aa, Homo sapiens
GGSRWQRGFPQLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 7 - protein, 221 aa, Homo sapiens
XQRGFPQLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMVSGRGRRACSLGCPFRGALC

SEQ ID NO: 8 - protein, 99 aa, Homo sapiens
MCKPATDPRFTRSPLAGGVILGVALWLRHDPQTTNLLYLELGDKPAPNTFYVGIYILIAVGAVMMFVGFLGCYGAIQESQCLLGTFFTCLVILFACEVA

SEQ ID NO: 9 - protein, 92 aa, Homo sapiens
MMFVGFLGCYGAIQESQCLLGTFFTCLVILFACEVAAGIWGFVNKDQIAKDVKQFYDQALQQAVVDDDANNAKAVVKTFHETLDCCGSSTLT

SEQ ID NO: 10 - protein, 51 aa, Homo sapiens
MMFVGFLGCYGAIQESQCLLGTFFTCLVILFACEVAAGIWGFVNKDQIAKD

SEQ ID NO: 11 - protein, 165 aa, Homo sapiens
MMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 12 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 13 - protein, 56 aa, Homo sapiens
MACLGFQRHKAQLNLATRTWPCTLLFFLLFIPVFCKAPPTEPECEKQFQPYFIPIN

SEQ ID NO: 14 - protein, 58 aa, Homo sapiens
MACLGFQRHKAQLNLATRTWPCTLLFFLLFIPVFCKAKEKKPSYNRGLCENAPNRARM

SEQ ID NO: 15 - protein, 79 aa, Homo sapiens
MACLGFQRHKAQLNLATRTWPCTLLFFLLFIPVFCKAMHVAQPAVVLASSRGIASFVSKEKKPSYNRGLCENAPNRARM

SEQ ID NO: 16 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 17 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 18 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 19 - protein, 178 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGD

SEQ ID NO: 20 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 21 - protein, 183 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKGQMEPRTHPTWLLHIFIPFCIIAFIFIATVIALRKQLCQKLYSSKDTTKRPVTTTKREVNSAI

SEQ ID NO: 22 - protein, 182 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKDGTQDPSNLAASHFHPLLHHCFHFHSHSDSPKKTTLSKAVFFKRHNKKTCHHNKEGSEQCYL

SEQ ID NO: 23 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 24 - protein, 66 aa, Homo sapiens
MQIPQAPWPVVWAVLQLGWRPGWFLDSPDRPWNPPTFSPALLVVTEGDNATFTCSFSNTSESFVLN

SEQ ID NO: 25 - protein, 122 aa, Homo sapiens
XNTSESFVLNWYRMSPSNQTDKLAAFPEDRSQPGQDCRFRVTQLPNGRDFHMSVVRARRNDSGTYLCGAISLAPKAQIKESLRAELRVTGTIGARRTGQPLKEDPSAVPVFSVDYGELDFQW